(19) United States
(12) Patent Application Publication
(10) Pub. No.: US 2025/0011285 A1
(43) Pub. Date: Jan. 9, 2025

(54) CYP11A1 INHIBITORS

(71) Applicant: ORION CORPORATION, Espoo (FI)

(72) Inventors: David DIN BELLE, Espoo (FI); Pekka PIETIKÄINEN, Espoo (FI); Petteri RUMMAKKO, Espoo (FI); Gerd WOHLFAHRT, Braunschweig (DE)

(21) Appl. No.: 18/708,157

(22) PCT Filed: Nov. 9, 2022
(86) PCT No.: PCT/FI2022/050741

§ 371 Date: May 7, 2024

(30) Foreign Application Priority Data
Nov. 10, 2021 (FI) 20217169

(51) Int. Cl. C07D 209/44; A61K 31/4035; A61K 31/423; A61K 31/454; A61K 31/4709; A61K 45/06; C07D 209/46; C07D 401/06; C07D 401/12; C07D 401/14; C07D 413/06; C07D 413/14

(52) U.S. Cl. CPC C07D 209/44; A61K 31/4035; A61K 31/423; A61K 31/454; A61K 31/4709; A61K 45/06; C07D 209/46; C07D 401/06; C07D 401/12; C07D 401/14; C07D 413/06; C07D 413/14

(57) ABSTRACT

The present invention relates to a compound of formula (I) wherein A, B, R₁, R₂, R₃, R₄ and R₅ are as defined in claim 1, or pharmaceutically acceptable salts thereof are disclosed. The compounds of formula (I) possess utility as cytochrome P450 monooxygenase 11A1 (CYP11A1) inhibitors. The compounds are useful as medicaments in the treatment of steroid receptor, for example androgen receptor or estrogen receptor, dependent diseases and conditions, such as cancer including prostate cancer and breast cancer.

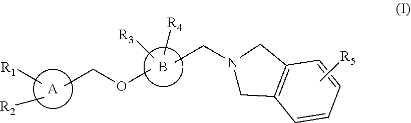

### TECHNICAL FIELD

The present invention relates to therapeutically active compounds useful in the treatment of a steroid receptor, such as androgen receptor (AR) or estrogen receptor (ER), dependent conditions and diseases, and to pharmaceutical compositions containing such compounds.

### BACKGROUND OF THE INVENTION

Treatments for steroid receptor dependent diseases such as androgen receptor (AR) dependent cancers and estrogen receptor (ER) dependent cancers have been investigated extensively. Prostate cancer, for example, is worldwide one of the most common cancers in men. Even though the 5-year survival rate of patients with localized prostate cancer is high, the prognosis for those patients, who develop castration-resistant prostate cancer (CRPC) within that 5-year follow-up period, is poor.

The androgen receptor (AR) signalling axis is critical in all stages of prostate cancer. In the CRPC stage (castration resistant prostate cancer), disease is characterized by high AR expression, AR amplification and persistent activation of the AR signalling axis by residual tissue/tumor androgens and by other steroid hormones and intermediates of steroid biosynthesis. Thus, treatment of advanced prostate cancer involves androgen deprivation therapy (ADT) such as hormonal manipulation using gonadotropin-releasing hormone (GnRH) agonists/antagonists or surgical castration, AR antagonists or CYP17A1 inhibitors (such as abiraterone acetate in combination with prednisone).

Although therapies can initially lead to disease regression, eventually majority of the patients develop a disease that is refractory to currently available therapies. Increased progesterone levels in patients treated with abiraterone acetate has been hypothesized to be one of the resistance mechanisms. Several nonclinical and clinical studies have indicated upregulation of enzymes that catalyse steroid biosynthesis at the late stage of CRPC. Very recently it has been published that 11β-OH androstenedione can be metabolized into 11-ketotestosterone (11-K-T) and 11-ketodehydrotestosterone (11-K-DHT) which can bind and activate AR as efficiently as testosterone and dihydrotestosterone. It has been shown that these steroids are found in high levels in plasma and tissue in prostate cancer patients, suggesting their role as AR agonists in CRPC. Furthermore, it has been addressed that prostate cancer resistance to CYP17A1 inhibition may still remain steroid dependent and responsive to therapies that can further suppress de novo intratumoral steroid synthesis upstream of CYP17A1, such as by CYP11A1 inhibition therapy (Cai, C. et al, Cancer Res., 71(20), 6503-6513, 2011).

Cytochrome P450 monooxygenase 11A1 (CYP11A1), also called cholesterol side chain cleavage enzyme, is a mitochondrial monooxygenase which catalyses the conversion of cholesterol to pregnenolone, the precursor of all steroid hormones. By inhibiting CYP11A1, the key enzyme of steroid biosynthesis upstream of CYP17A1, the total block of the whole steroid biosynthesis can be achieved. CYP11A1 inhibitors may therefore have a great potential for treating steroid hormone dependent cancers, including prostate cancer, even in advanced stages of the disease, and especially in those patients who appear to be hormone refractory. It has been shown that a compound having CYP11A1 inhibitory effect significantly inhibited tumor growth in vivo in a murine CRPC xenograft model (Oksala, R. et al, Annals of Oncology, (2017) 28 (suppl. 5): Abstract/Poster 28P). CYP11A1 inhibitors have been described earlier in WO 2018/115591.

### SUMMARY OF THE INVENTION

It has been found that compounds of formula (I) are potent CYP11A1 inhibitors. The compounds of the invention are therefore particularly useful as medicaments in the treatment of steroid hormone dependent conditions and diseases where CYP11A1 inhibition is desired. Such conditions and diseases include, but are not limited to, endocrine cancers and diseases, such as prostate cancer and breast cancer. In particular, the compounds of the invention are useful in the treatment of AR dependent conditions and diseases including prostate cancer.

The present invention relates to a compound of formula (I) or a pharmaceutically acceptable salt thereof

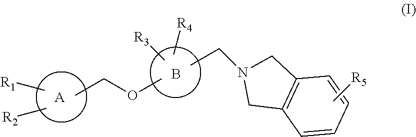

wherein
   A is a 3-10 membered carbocyclyl or a 4-12 membered heterocyclyl containing 1-4 heteroatoms selected from O, N or S;
   B is phenyl or a 8-12 membered heterocyclyl containing 1-4 heteroatoms selected from O, N or S;
   R₁ is hydrogen, halogen, C₁₋₇ alkyl, C₂₋₇ alkenyl, C₃₋₇ cycloalkyl, C₁₋₇ alkoxy, hydroxy, hydroxy C₁₋₇ alkyl, halogen C₁₋₇ alkyl, —X₁—C(O)R₆, —SO₂—R₇, —S(O)(═NH)(C₁₋₇ alkyl);
   with the proviso that when B is phenyl, then R₁ is not hydrogen;
   R₂ is hydrogen, C₁₋₇ alkyl, C₂₋₇ alkenyl, C₃₋₇ cycloalkyl, C₁₋₇ alkoxy, halogen or halogen C₁₋₇ alkyl;
   R₃ is hydrogen, C₁₋₇ alkyl, C₂₋₇ alkenyl, C₃₋₇ cycloalkyl, hydroxy C₁₋₇ alkyl, halogen C₁₋₇ alkyl, C₁₋₇ alkoxy C₁₋₇ alkyl, nitro, cyano, amino, halogen, —X₂—C(O)R₈, —X₃—NR₉R₁₀; —OR₁₁;
   R₄ is hydrogen, halogen or nitro;
   R₅ is hydrogen, halogen, halogen C₁₋₇ alkyl, —S—C₁₋₇ alkyl, —S(O)₂—C₁₋₇ alkyl or oxo;
   X₁, X₂ and X₃ are, independently, a bond, C₁₋₇ alkyl or C₂₋₇ alkenyl;
   R₆ is C₁₋₇ alkyl, C₁₋₇ alkoxy, —NR₁₂R₁₃;
   R₇ is C₁₋₇ alkyl, C₁₋₇ alkoxy C₁₋₇ alkyl, —NHR₁₄ or a 4-6 membered heterocyclyl optionally substituted with 1-3 C₁₋₇ alkyl groups;
   R₈ is C₁₋₇ alkoxy or —NR₁₅R₁₆;
   R₉ is hydrogen, C₁₋₇ alkyl, —C(O)—C₁₋₇ alkyl, —C(O)-halogen C₁₋₇ alkyl or —S(O)₂—C₁₋₇ alkyl;
   R₁₀ is hydrogen or C₁₋₇ alkyl:
   R₁ is hydrogen, C₁₋₇ alkyl, halogen C₁₋₇ alkyl or —C₁₋₇ alkyl-C(O)—NH₂;
   R₁₂ is hydrogen, C₁₋₇ alkyl, C₁₋₇ alkoxy C₁₋₇ alkyl, —C₁₋₇ alkyl-O—C(O)—C₁₋₇ alkyl;
   R₁₃ is hydrogen or C₁₋₇ alkyl;
   R₁₄ is C₁₋₇ alkyl or C₁₋₇ alkoxy C₁₋₇ alkyl; and
   R₁₅ and R₁₆ are, independently, hydrogen or C₁₋₇ alkyl.

According to one embodiment, the invention provides a method for the treatment of a steroid receptor dependent condition or disease comprising administering to a subject in need thereof a therapeutically effective amount of a compound of formula (I) or a pharmaceutically acceptable salt thereof

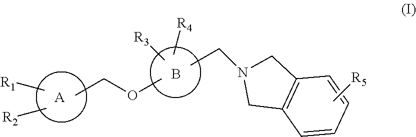

wherein
   A is a 3-10 membered carbocyclyl or a 4-12 membered heterocyclyl containing 1-4 heteroatoms selected from O, N or S;
   B is phenyl or a 8-12 membered heterocyclyl containing 1-4 heteroatoms selected from O, N or S;
   R₁ is hydrogen, halogen, C₁₋₇ alkyl, C₂₋₇ alkenyl, C₃₋₇ cycloalkyl, C₁₋₇ alkoxy, hydroxy, hydroxy C₁₋₇ alkyl, halogen C₁₋₇ alkyl, —X₁—C(O)R₆, —SO₂—R₇, —S(O)(═NH)(C₁₋₇ alkyl);
   with the proviso that when B is phenyl, then R₁ is not hydrogen;
   R₂ is hydrogen, C₁₋₇ alkyl, C₂₋₇ alkenyl, C₃₋₇ cycloalkyl, C₁₋₇ alkoxy, halogen or halogen C₁₋₇ alkyl;
   R₃ is hydrogen, C₁₋₇ alkyl, C₂₋₇ alkenyl, C₃₋₇ cycloalkyl, hydroxy C₁₋₇ alkyl, halogen C₁₋₇ alkyl, C₁₋₇ alkoxy C₁₋₇ alkyl, nitro, cyano, amino, halogen, —X₂—C(O)R₈, —X₃—NR₉R₁₀; —OR₁₁;
   R₄ is hydrogen, halogen or nitro;
   R₅ is hydrogen, halogen, halogen C₁₋₇ alkyl, —S—C₁₋₇ alkyl, —S(O)₂—C₁₋₇ alkyl or oxo;
   X₁, X₂ and X₃ are, independently, a bond, C₁₋₇ alkyl or C₂₋₇ alkenyl;
   R₆ is C₁₋₇ alkyl, C₁₋₇ alkoxy, —NR₁₂R₁₃;
   R₇ is C₁₋₇ alkyl, C₁₋₇ alkoxy C₁₋₇ alkyl, —NHR₁₄ or a 4-6 membered heterocyclyl optionally substituted with 1-3 C₁₋₇ alkyl groups;
   R₈ is C₁₋₇ alkoxy or —NR₁₅R₁₆;
   R₉ is hydrogen, C₁₋₇ alkyl, —C(O)—C₁₋₇ alkyl, —C(O)-halogen C₁₋₇ alkyl or —S(O)₂—C₁₋₇ alkyl;
   R₁₀ is hydrogen or C₁₋₇ alkyl:
   R₁ is hydrogen, C₁₋₇ alkyl, halogen C₁₋₇ alkyl or —C₁₋₇ alkyl-C(O)—NH₂;
   R₁₂ is hydrogen, C₁₋₇ alkyl, C₁₋₇ alkoxy C₁₋₇ alkyl, —C₁₋₇ alkyl-O—C(O)—C₁₋₇ alkyl;
   R₁₃ is hydrogen or C₁₋₇ alkyl;
   R₁₄ is C₁₋₇ alkyl or C₁₋₇ alkoxy C₁₋₇ alkyl; and
   R₁₅ and R₁₆ are, independently, hydrogen or C₁₋₇ alkyl.

According to one embodiment, the steroid receptor dependent conditions or diseases include, but are not limited to, endocrine cancers and diseases, such as prostate cancer, particularly castration resistant prostate cancer (CRPC), and breast cancer.

According to one embodiment, the invention provides a pharmaceutical composition comprising a compound of formula (I) together with a pharmaceutically acceptable carrier.

### DETAILED DESCRIPTION OF THE INVENTION

The present application provides novel compounds of formula (I) or pharmaceutically acceptable salts thereof which are useful as CYP11A1 inhibitors.

One of the embodiments of the present invention provides a compound of formula (I) or a pharmaceutically acceptable salt thereof

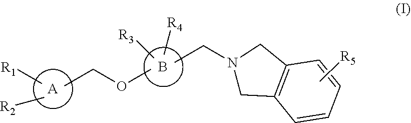

wherein
   A is a 3-10 membered carbocyclyl or a 4-12 membered heterocyclyl containing 1-4 heteroatoms selected from O, N or S;
   B is phenyl or a 8-12 membered heterocyclyl containing 1-4 heteroatoms selected from O, N or S;
   R₁ is hydrogen, halogen, C₁₋₇ alkyl, C₂₋₇ alkenyl, C₃₋₇ cycloalkyl, C₁₋₇ alkoxy, hydroxy, hydroxy C₁₋₇ alkyl, halogen C₁₋₇ alkyl, —X₁—C(O)R₆, —SO₂—R₇, —S(O)(═NH)(C₁₋₇ alkyl);
   with the proviso that when B is phenyl, then R₁ is not hydrogen;
   R₂ is hydrogen, C₁₋₇ alkyl, C₂₋₇ alkenyl, C₃₋₇ cycloalkyl, C₁₋₇ alkoxy, halogen or halogen C₁₋₇ alkyl;
   R₃ is hydrogen, C₁₋₇ alkyl, C₂₋₇ alkenyl, C₃₋₇ cycloalkyl, hydroxy C₁₋₇ alkyl, halogen C₁₋₇ alkyl, C₁₋₇ alkoxy C₁₋₇ alkyl, nitro, cyano, amino, halogen, —X₂—C(O)R₈, —X₃—NR₉R₁₀; —OR₁₁;
   R₄ is hydrogen, halogen or nitro;
   R₅ is hydrogen, halogen, halogen C₁₋₇ alkyl, —S—C₁₋₇ alkyl, —S(O)₂—C₁₋₇ alkyl or oxo;
   X₁, X₂ and X₃ are, independently, a bond, C₁₋₇ alkyl or C₂₋₇ alkenyl;
   R₆ is C₁₋₇ alkyl, C₁₋₇ alkoxy, —NR₁₂R₁₃;
   R₇ is C₁₋₇ alkyl, C₁₋₇ alkoxy C₁₋₇ alkyl, —NHR₁₄ or a 4-6 membered heterocyclyl optionally substituted with 1-3 C₁₋₇ alkyl groups;
   R₈ is C₁₋₇ alkoxy or —NR₁₅R₁₆;
   R₉ is hydrogen, C₁₋₇ alkyl, —C(O)—C₁₋₇ alkyl, —C(O)-halogen C₁₋₇ alkyl or
   —S(O)₂—C₁₋₇ alkyl;
   R₁₀ is hydrogen or C₁₋₇ alkyl:
   R₁₁ is hydrogen, C₁₋₇ alkyl, halogen C₁₋₇ alkyl or —C₁₋₇ alkyl-C(O)—NH₂;
   R₁₂ is hydrogen, C₁₋₇ alkyl, C₁₋₇ alkoxy C₁₋₇ alkyl, —C₁₋₇ alkyl-O—C(O)—C₁₋₇ alkyl;
   R₁₃ is hydrogen or C₁₋₇ alkyl;
   R₁₄ is C₁₋₇ alkyl or C₁₋₇ alkoxy C₁₋₇ alkyl; and
   R₁₅ and R₁₆ are, independently, hydrogen or C₁₋₇ alkyl.

According to one embodiment, specifically provided is a compound according to formula (I) wherein B is phenyl or any one of the following groups:

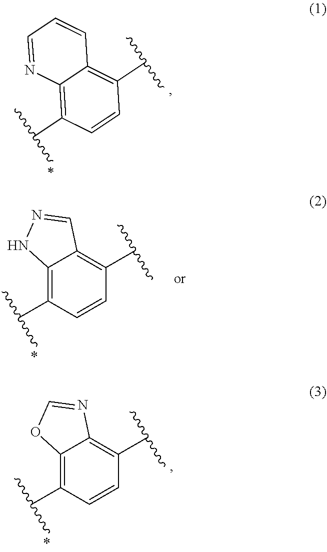 [SMILES: *c1ccc(*)c2ncccc12 ; *c1ccc(*)c2[nH]ncc12 ; *c1ccc(*)c2ocnc12]

wherein the asterisk denotes the point of attachment to oxygen atom.

According one embodiment, B is phenyl, or as another embodiment B is ring (1), or as another embodiment B is ring (2), or as another embodiment B is ring (3).

According to yet one embodiment, specifically provided are compounds according to any of the above embodiments wherein A is a 5-6 membered carbocyclyl or a 5-6 membered heterocyclyl containing 1-2 heteroatoms selected from O, N or S.

According to yet one embodiment, specifically provided are compounds according to any of the above embodiments wherein A is phenyl, cyclohexyl or piperidinyl. In a subgroup of the preceding embodiment, A is phenyl or piperidinyl.

According to yet one embodiment, specifically provided are compounds according to any of the above embodiments wherein R₁ is halogen, C₁₋₇ alkyl, C₂₋₇ alkenyl, C₃₋₇ cycloalkyl, C₁₋₇ alkoxy, hydroxy, hydroxy C₁₋₇ alkyl, halogen C₁₋₇ alkyl, —X₁—C(O)R₆, —SO₂—R₇ or —S(O)(═NH)(C₁₋₇ alkyl). In a subgroup of the preceding embodiment R₁ is —X₁—C(O)R₆, —SO₂—R₇ or —S(O)(═NH)(C₁₋₇ alkyl). In yet another subgroup of the preceding embodiment, R₁ is —X₁—C(O)R₆.

According to one embodiment, specifically provided are compounds according to any of the above embodiments wherein X₁ is a bond. According to yet one embodiment, specifically provided are compounds according to any of the above embodiments wherein R₆ is C₁₋₇ alkyl, C₁₋₇ alkoxy or —NR₁₂R₁₃ and R₁₂ is C₁₋₇ alkyl, C₁₋₇ alkoxy C₁₋₇ alkyl or —C₁₋₇ alkyl-O—C(O)—C₁₋₇ alkyl.

According to another embodiment, specifically provided are compounds according to any of the above embodiments wherein R₁ is —SO₂—R₇. According to another embodiment, specifically provided are compounds according to any of the above embodiments wherein R₇ is C₁₋₇ alkyl, C₁₋₇ alkoxy C₁₋₇ alkyl or —NHR₁₄.

In one aspect, provided are compounds according to any of the above embodiments wherein R₂ is hydrogen. In one aspect, provided are compounds according to any of the above embodiments wherein R₄ is hydrogen or halogen.

According to another embodiment, specifically provided are compounds according to any of the above embodiments wherein R₃ is hydrogen, C₁₋₇ alkyl, C₂₋₇ alkenyl, C₃₋₇ cycloalkyl, hydroxy C₁₋₇ alkyl, halogen C₁₋₇ alkyl, C₁₋₇ alkoxy C₁₋₇ alkyl, nitro, cyano, halogen, —X₂—C(O)R₈ or —OR₁₁. In a subclass of the above embodiment are compounds wherein R₃ is hydrogen, C₃₋₇ cycloalkyl, hydroxy C₁₋₇ alkyl, halogen C₁₋₇ alkyl, C₁₋₇ alkoxy C₁₋₇ alkyl, nitro, cyano, —X₂—C(O)R₈ or —OR₁₁. In one aspect, provided are compounds according to any of the above embodiments wherein X₂ is a bond.

According to another embodiment, specifically provided are compounds according to any of the above embodiments wherein R₈ is C₁₋₇ alkoxy. In one aspect, provided are compounds according to any of the above embodiments wherein Rn is C₁₋₇ alkyl or —C₁₋₇ alkyl-C(O)—NH₂.

According to another embodiment, specifically provided are compounds according to any of the above embodiments wherein R₅ is hydrogen, halogen C₁₋₇ alkyl or —S—C₁₋₇ alkyl.

According to still one embodiment, the present invention provides a method for the treatment of a steroid receptor dependent conditions and diseases, comprising administering to a subject in need thereof a therapeutically effective amount of a compound of formula (I) as defined in any of the above embodiments.

According to one embodiment, the steroid receptor dependent disease or condition is androgen receptor or estrogen receptor dependent disease or condition including endocrine cancers and diseases, for example prostate cancer or breast cancer, particularly castration-resistant prostate cancer (CRPC), for example metastatic castration resistant prostate cancer (mCRPC) or non-metastatic castration resistant prostate cancer (nmCRPC). According to one embodiment of the invention, the CRPC to be treated is refractory to CYP17A1 inhibitor treatment. According to another embodiment, the androgen receptor dependent disease or condition is endocrine cancer which is dependent upon CYP11A1 activation.

The compounds of the invention can be prepared by a variety of synthetic routes analogously to the methods known in the literature using suitable starting materials. The compounds according to formula (I) can be prepared e.g. analogously or according to the following reaction Schemes. Some compounds included in the formula (I) can be obtained by converting the functional groups of the other compounds of formula (I) obtained in accordance with the following Schemes, by well known reaction steps such as oxidation, reduction, hydrolysis, acylation, alkylation, amidation, amination, sulfonation and others. It should be noted that any appropriate leaving groups, e.g. N-protecting groups, such as a t-butoxycarbonyl (t-BOC) group or a phenylsulfonyl group, can be used in well known manner during the syntheses in order to improve the selectivity of the reaction steps.

Compounds of formula (I) can be prepared, for example, by reductive amination according to Scheme 1, wherein A, B, R₁, R₂, R₃, R₄ and R₅ are as defined above. In the method of Scheme 1, the aldehyde compound of formula [1] is reacted with the isoindoline compound of formula [2] in a suitable solvent such as dimethylformamide dichloromethane or 1,2-dichloroethane in the presence of acetic acid and a reducing agent such as sodium triacetoxy borohydride (STAB) to produce a compound of formula (I).

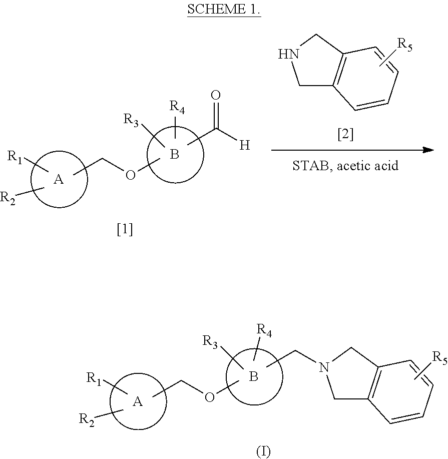

Alternatively, compounds of formula (I) can be prepared, for example, according to Scheme 2, wherein A, B, R₁, R₂, R₃, R₄, R₅, R₂₃, R₂₄, L, A and B, are as defined above, and X is a halogen. In the method of Scheme 2, the compound of formula [3] is coupled with ring A derivative [4] where halogen is acting as the leaving group in a suitable solvent in the presence of a base at elevated temperature, for example using K₂CO₃ in DMSO or DMF, to produce a compound of formula (I).

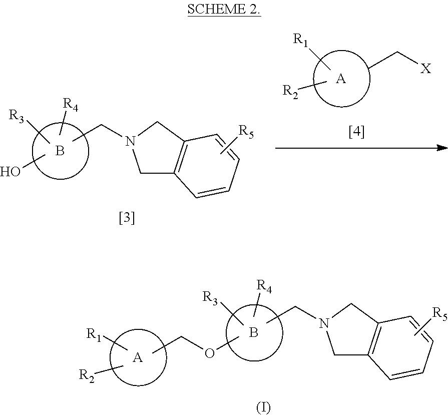

Alternatively, compounds of formula (I) can be prepared according to Scheme 3, wherein A, B, R₁, R₂, R₃, R₄, R₅ are as defined above, and Z is mesyl or tosyl group. In the method of Scheme 3, the compound of formula [5] is coupled with ring A derivative [6], where mesylate or tosylate is acting as the leaving group, in a suitable solvent in the presence of a base at elevated temperature, for example using tris(2-(2-methoxyethoxy)-ethyl)amine and/or Cs₂CO₃ in DMSO or K₂CO₃ in DMSO or DMF to produce a compound of formula (I).

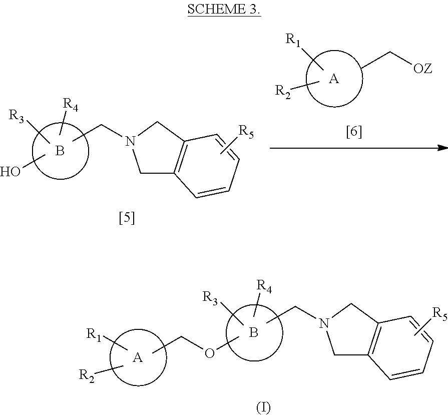

Intermediate compounds can be prepared according to the methods disclosed in the literature or as disclosed in the present disclosure.

For example, intermediate compounds of formula [1] can be prepared according to Scheme 4, wherein A, B, R₁, R₂, R₃ and R₄ are as defined above, and X and is a halogen, preferably chloro or bromo. In the method of Scheme 4, a compound of formula [7] is coupled with a compound of formula [8] in a suitable solvent such as DMF in the presence of a base such as K₂CO₃ together with potassium iodide to produce a compound of formula [la].

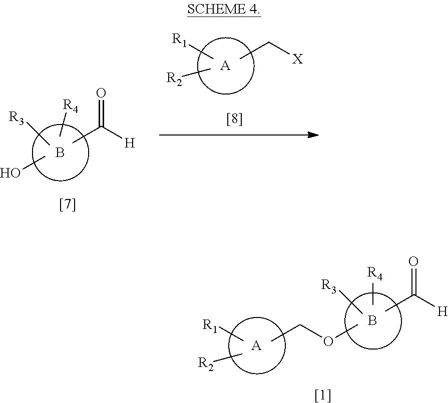

Alternatively, the intermediate compounds or compounds of formula (I) can be prepared as disclosed in the specific Examples of the present disclosure.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “subject”, as employed herein, refers to humans and animals.

The term “steroid receptor” refers to receptor which binds to and is activated by a steroid hormone. Examples of steroid receptors include, but are not limited to, androgen, estrogen, glucocorticoid, and progesterone receptors.

The term “endocrine cancer” refers to partially or completely unregulated growth of one or more cellular components of the endocrine system, including, but not limited to, cancers of one or more of the adrenal glands.

The term “elevated temperature” refers to a temperature higher than room temperature, typically from about 30 to about 120° C., from example from about 40 to about 100° C. or from about 50 to about 80° C.

The term “halo” or “halogen”, as employed herein as such or as part of another group, refers to chlorine, bromine, fluorine or iodine.

The term “C₁₋₇ alkyl”, as employed herein as such or as part of another group, refers to a straight or branched chain saturated hydrocarbon group having 1, 2, 3, 4, 5, 6 or 7 carbon atom(s). Representative examples of C₁₋₇ alkyl include, but are not limited to, methyl, ethyl, n-propyl, iso-propyl, n-butyl, iso-butyl, sec-butyl, tert-butyl, n-pentyl, iso-pentyl and n-hexyl. One preferred embodiment of “C₁₋₇ alkyl” is C₁₋₃ alkyl. The term “C₁₋₃ alkyl” refers to a preferred embodiment of “C₁₋₇ alkyl” having 1, 2 or 3 carbon atoms.

The term “C₂₋₇ alkenyl”, as employed herein as such or as part of another group, refers to an aliphatic hydrocarbon group having 2, 3, 4, 5, 6 or 7 carbon atoms and containing one or several double bonds. Representative examples include, but are not limited to, ethenyl, propenyl and cyclohexenyl.

The term “C₃₋₇ cycloalkyl”, as employed herein as such or as part of another group, refers to a saturated cyclic hydrocarbon group containing 3, 4, 5, 6 or 7 carbon atoms. Representative examples of cycloalkyl include, but are not limited to, cyclo-propyl, cyclobutyl, cyclopentyl and cyclohexyl.

The term “hydroxy”, as employed herein as such or as part of another group, refers to an —OH group.

The term “cyano”, as employed herein as such or as part of another group, refers to a —CN group.

The term “carboxy”, as employed herein as such or as part of another group, refers to —COOH group.

The term “carbonyl”, as employed herein as such or as part of another group, refers to a carbon atom double-bonded to an oxygen atom (C═O).

The term “oxo”, as employed herein as such or as part of another group, refers to oxygen atom linked to another atom by a double bond (═O).

The term “C₁₋₇ alkoxy”, as employed herein as such or as part of another group, refers to C₁₋₇ alkyl, as defined herein, appended to the parent molecular moiety through an oxygen atom. Representative examples of C₁₋₇ alkoxy include, but are not limited to methoxy, ethoxy, propoxy, butoxy, isobutoxy, sec-butoxy and tert-butoxy.

The term “hydroxy C₁₋₇ alkyl”, as employed herein, refers to at least one hydroxy group, as defined herein, appended to the parent molecular moiety through a C₁₋₇ alkyl group, as defined herein. Representative examples of hydroxy C₁₋₇ alkyl include, but are not limited to, hydroxymethyl, 2,2-dihydroxyethyl, 1-hydroxyethyl, 3-hydroxypropyl, 1-hydroxypropyl, 1-methyl-1-hydroxyethyl and 1-methyl-1-hydroxypropyl.

The term “halogen C₁₋₇ alkyl”, as employed herein, refers to at least one halogen, as defined herein, appended to the parent molecular moiety through a C₁₋₇ alkyl group, as defined herein. Representative examples of halo C₁₋₇ alkyl include, but are not limited to, fluoromethyl, difluoromethyl, trifluoromethyl, 2-chloroethyl and 3-bromopropyl.

The term “cyano C₁₋₇ alkyl”, as employed herein, refers to a cyano group, as defined herein, appended to the parent molecular moiety through a C₁₋₇ alkyl group, as defined herein. Representative examples of cyano C₁₋₇ alkyl include, but are not limited to, cyanomethyl, 1-cyanoethyl, 1-cyanopropyl and 2-cyanopropyl.

The term “halogen C₁₋₇ alkoxy”, as employed herein, refers to at least one halogen, as defined herein, appended to the parent molecular moiety through a C₁₋₇ alkoxy group, as defined herein.

The term “phenyl C₁₋₇ alkyl”, as employed herein, refers to at least one phenyl group appended to the parent molecular moiety through a C₁₋₇ alkyl group, as defined herein.

The term “C₁₋₇ alkyl carbonyl”, as employed herein as such or as part of another group, refers to a C₁₋₇ alkyl group, as defined herein, appended to the parent molecular moiety through a carbonyl group, as defined herein.

The term “C₁₋₇ alkoxy C₁₋₇ alkyl”, as employed herein as such or as part of another group, refers to at least one C₁₋₇ alkoxy group, as defined herein, appended to the parent molecular moiety through an C₁₋₇ alkyl group, as defined herein.

The term “4-12 membered heterocyclyl” as employed herein, refers to a saturated, partially saturated or aromatic ring with 4-12 ring atoms, of which 1-4 atoms are heteroatoms selected from a group consisting of N, O and S. One embodiment of a “4-12 membered heterocyclyl” is a “4-10 membered heterocyclyl” which refers to a saturated, partially saturated or aromatic ring with 4-10 ring atoms, of which 1-4 atoms are heteroatoms selected from a group consisting of N, O and S. Representative examples of a 4-12 membered heterocyclyl include, but are not limited to, oxetanyl, azetidinyl, pyrazolyl, 1,2,4-triazol-1-yl, 1,2,3-triazol-1-yl, pyrimidinyl, pyridinyl, piperidinyl, tetrazolyl, piperazinyl, furanyl, morpholinyl, pyrrolidinyl, thiazolyl, isoxazolyl, pyrazinyl, tetrahydropyranyl, 1,2,4-oxadiazolyl, oxazolyl, imidazolyl, indolyl, 4,5-dihydroimidazolyl, indazolyl, quinolinyl, benzoxazolyl, benzofuranyl, benzimidazolyl, benzothiazolyl and indolyl rings.

The term “8-12 membered heterocyclyl” as employed herein, refers to a saturated, partially saturated or aromatic ring with 8-12 ring atoms, of which 1-4 atoms are heteroatoms selected from a group consisting of N, O and S. Representative examples of a 8-12 membered heterocyclyl include, but are not limited to, indazolyl, quinolinyl, benzoxazolyl, benzofuranyl, benzimidazolyl, benzothiazolyl, indolyl, 1H-benzo[d][1,2,3]triazolyl, pyrazolo[1,5-a]pyrimidinyl, benzo[d]imidazolyl, imidazo[4,5-b]pyridinyl and 4,5,6,7-tetrahydrobenzo[d]imidazolyl rings.

The term “4-6 membered heterocyclyl” as employed herein, refers to a saturated, partially saturated or aromatic ring with 4-6 ring atoms, of which 1-4 atoms are heteroatoms selected from a group consisting of N, O and S. Representative examples of a 4-6 membered heterocyclyl include, but are not limited to, oxetanyl, azetidinyl, pyrazolyl, 1,2,4-triazol-1-yl, 1,2,3-triazol-1-yl, pyrimidinyl, pyridinyl, piperidinyl, tetrazolyl, piperazinyl, furanyl, morpholinyl, pyrrolidinyl, thiazolyl, isoxazolyl, pyrazinyl, tetrahydropyranyl, 1,2,4-oxadiazolyl, oxazolyl, imidazolyl, indolyl and 4,5-dihydroimidazolyl rings.

The term “3-10 membered carbocyclyl”, as employed herein, refers to a saturated, partially saturated or aromatic ring with 3 to 10 ring atoms consisting of carbon atoms only. One embodiment of a “3-10 membered carbocyclyl” is a “3-6 membered carbocyclyl” which refers to a saturated, partially saturated or aromatic ring with 3 to 6 ring atoms consisting of carbon atoms only. Representative examples of a 3-10 membered carbocyclyl group include, but are not limited to, phenyl, cyclohexyl, cyclohexenyl, cyclopentyl, cyclopentenyl and cyclobutyl rings.

The term “substituted” as used herein in connection with various residues refers to, if not otherwise defined, to halogen substituents, such as fluorine, chlorine, bromine, iodine, or C₁₋₇ alkyl, C₃₋₇ cycloalkyl, hydroxy, amino, nitro, cyano, thiol C₁₋₇ alkyl, methylsulfonyl, C₁₋₇ alkoxy, halo C₁₋₇ alkyl, hydroxy C₁₋₇ alkyl or amino C₁₋₇ alkyl substituents. Preferred are halogen, C₁₋₇ alkyl, hydroxy, amino, halo C₁₋₇ alkyl, C₁₋₇ alkoxy and methylsulfonyl substituents. In one group of preferred substituents are 1-2 substituents selected from C₁₋₇ alkyl or halogen substituents, particularly C₁₋₃ alkyl or halogen substituents, particularly methyl, ethyl, chloro, fluoro or bromo substituents.

The “substituted” groups may contain 1 to 3, preferably 1 or 2, of the above mentioned substituents, if not otherwise defined.

Optically active enantiomers or diastereomers of compounds of formula (I) can be prepared e.g. by resolution of the racemic end product by known methods or by using suitable optically active starting materials. Similarly, racemic compounds of formula (I) can be prepared by using racemic starting materials. Resolution of racemic compounds of formula (I) or a racemic starting material thereof can be carried out, for example, by converting the racemic compound into its diastereromeric salt mixture by reaction with an optically active acid and subsequent separation of the diastereomers by crystallization. Representative examples of said optically active acids include, but are not limited to, D-tartaric acid and dibenzoyl-D-tartaric acid. Alternatively, preparative chiral chromatography may be used for resolution of the racemic mixture.

Pharmaceutically acceptable salts are well known in the field of pharmaceuticals. Non-limiting examples of suitable salts include metal salts, ammonium salts, salts with an organic base, salts with an inorganic acid, salts with organic acid, and salts with basic or acidic amino acid. Non-limiting examples of metal salts include alkali metal salts such as sodium salt and potassium salt; alkaline earth metal salts such as calcium salt, and magnesium salt. Non-limiting examples of salts with inorganic or organic acids include chlorides, bromides, sulfates, nitrates, phosphates, sulfonates, methane sulfonates, formates, tartrates, maleates, citrates, benzoates, salicylates, ascorbates, acetates, oxalates, fumarates, hemifumarates, and succinates. Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols, e.g. methyl, ethyl, propyl, isopropyl, butyl, isobutyl, sec-butyl, tert-butyl esters. Phosphate esters and carbonate esters, are also within the scope of the invention.

The definition of formula (I) above is inclusive of all the possible isotopes and isomers, such as stereoisomers, of the compounds, including geometric isomers, for example Z and E isomers (cis and trans isomers), and optical isomers, e.g. diastereomers and enantiomers, and prodrug esters, e.g. phosphate esters and carbonate esters.

It will be appreciated by those skilled in the art that the present compounds may contain at least one chiral center. Accordingly, the compounds may exist in optically active or racemic forms. It is to be understood that the formula (I) includes any racemic or optically active form, or mixtures thereof. In one embodiment, the compounds are the pure (R)-isomers. In another embodiment, the compounds are the pure (S)-isomers. In another embodiment, the compounds are a mixture of the (R) and the (S) isomers. In another embodiment, the compounds are a racemic mixture comprising an equal amount of the (R) and the (S) isomers. The compounds may contain two chiral centers. In such case, according to one embodiment, the compounds are a mixture of diasteromers.

According to another embodiment, the compounds of the invention are a mixture of enantiomers. According to still another embodiment, the compounds are pure enantiomers. The individual isomers may be obtained using the corresponding isomeric forms of the starting material or they may be separated after the preparation of the end compound according to conventional separation methods. For the separation of optical isomers, e.g. enantiomers or diastereomers, from the mixture thereof the conventional resolution methods, e.g. fractional crystallisation, may be used.

The present compounds may also exist as tautomers or equilibrium mixtures thereof wherein a proton of a compound shifts from one atom to another. Examples of tautomerism include, but are not limited to, amido-imido, keto-enol, phenol-keto, oxime-nitroso, nitro-aci, imine-enamine, annular tautomerism of heterocyclic rings such as pyrazole ring, and the like. Tautomeric forms are intended to be encompassed by compounds of formula (I), even though only one tautomeric form may be depicted.

Examples of preferred compounds of one group of formula (I) include
Methyl 4-((4-(isoindolin-2-ylmethyl)phenoxy)methyl)benzoate (Compound 2);
4-((4-(Isoindolin-2-ylmethyl)-2-methoxyphenoxy)methyl)-N,N-dimethyl-benzamide (Compound 3);
4-((2-Chloro-4-(isoindolin-2-ylmethyl)phenoxy)methyl)-N,N-dimethyl-benzamide (Compound 4);
4-((2-Hydroxy-4-(isoindolin-2-ylmethyl)phenoxy)methyl)-N,N-dimethyl-benzamide (Compound 5);
4-((2-Cyano-4-(isoindolin-2-ylmethyl)phenoxy)methyl)-N,N-dimethylbenzamide (Compound 6);
Methyl 2-((4-(dimethylcarbamoyl)benzyl)oxy)-5-(isoindolin-2-ylmethyl)-benzoate (Compound 7);
4-((2-Ethoxy-4-(isoindolin-2-ylmethyl)phenoxy)methyl)-N,N-dimethyl-benzamide (Compound 8);
Methyl (E)-3-(4-((4-(isoindolin-2-ylmethyl)-2-methoxyphenoxy)methyl)phenyl)acrylate (Compound 9);
4-((4-(Isoindolin-2-ylmethyl)-2-(methoxymethyl)phenoxy)methyl)-N,N-di-methylbenzamide (Compound 10);
4-((4-(Isoindolin-2-ylmethyl)-2-(2,2,2-trifluoroethoxy)phenoxy)methyl)-N,N-dimethylbenzamide (Compound 11);
2-(3-Methoxy-4-((4-(methylsulfonyl)benzyl)oxy)benzyl)isoindoline (Compound 12);
4-((4-(Isoindolin-2-ylmethyl)-2-methoxyphenoxy)methyl)-N-(2-methoxyethyl)-benzenesulfonamide (Compound 13);
4-((2-(2-Amino-2-oxoethoxy)-4-(isoindolin-2-ylmethyl)phenoxy)methyl)-N,N-dimethylbenzamide (Compound 14);
4-((2-Allyl-4-(isoindolin-2-ylmethyl)phenoxy)methyl)-N,N-dimethylbenzamide (Compound 15);
5-(Isoindolin-2-ylmethyl)-2-((4-(methylsulfonyl)benzyl)oxy)benzonitrile (Compound 16);
5-(Isoindolin-2-ylmethyl)-N,N-dimethyl-2-((4-(methylsulfonyl)benzyl)oxy)-benzamide (Compound 17);
2-(3-(Methoxymethyl)-4-((4-(methylsulfonyl)benzyl)oxy)benzyl)isoindoline (Compound 18);
5-(Isoindolin-2-ylmethyl)-2-((1-(methylsulfonyl)piperidin-4-yl)methoxy)-benzonitrile (Compound 19);
2-(4-((4-((2-Methoxyethyl)sulfonyl)benzyl)oxy)-3-(methoxymethyl)benzyl)-isoindoline (Compound 20);
2-(2-Fluoro-3-methoxy-4-((4-(methylsulfonyl)benzyl)oxy)benzyl)isoindoline (Compound 21);
2-(3-(Methoxymethyl)-4-((1-(methylsulfonyl)piperidin-4-yl)methoxy)benzyl)-isoindoline (Compound 22);
4-((4-(Isoindolin-2-ylmethyl)-2-methoxy-3-nitrophenoxy)methyl)-N-isopropyl-piperidine-1-carboxamide (Compound 23);
4-((3-Fluoro-4-(isoindolin-2-ylmethyl)phenoxy)methyl)-N,N-dimethylbenzamide (Compound 24);
4-((3,5-Difluoro-4-(isoindolin-2-ylmethyl)phenoxy)methyl)-N,N-dimethyl-benzamide (Compound 25);
4-((2,5-Difluoro-4-(isoindolin-2-ylmethyl)phenoxy)methyl)-N,N-dimethyl-benzamide (Compound 26);
4-((2,3-Difluoro-4-(isoindolin-2-ylmethyl)phenoxy)methyl)-N,N-dimethyl-benzamide (Compound 27);
4-((2-Fluoro-4-(isoindolin-2-ylmethyl)-6-methoxyphenoxy)methyl)-N,N-di-methylbenzamide (Compound 28);
4-((4-(Isoindolin-2-ylmethyl)-2-(trifluoromethoxy)phenoxy)methyl)-N,N-dimethylbenzamide (Compound 29); N,N-Dimethyl-4-((4-((1-oxoisoindolin-2-yl)methyl)-2-(trifluoromethoxy)-phenoxy)methyl)benzamide (Compound 30);
4-((4-(Isoindolin-2-ylmethyl)-3-methoxyphenoxy)methyl)-N,N-dimethyl-benzamide (Compound 31);
4-((2-Methoxy-4-((5-(methylsulfonyl)isoindolin-2-yl)methyl)phenoxy)methyl)-N,N-dimethylbenzamide (Compound 32);
5-(isoindolin-2-ylmethyl)-2-((4-(S-methylsulfonimidoyl)benzyl)oxy)benzonitrile (Compound 33);
4-((2-Methoxy-4-((5-(methylthio)isoindolin-2-yl)methyl)phenoxy)methyl)-N,N-dimethylbenzamide (Compound 34);
4-((4-(Isoindolin-2-ylmethyl)-2-methoxyphenoxy)methyl)-N-(2-methoxyethyl)-N-methylbenzamide (Compound 35);
2-(4-((4-(Isoindolin-2-ylmethyl)-2-methoxyphenoxy)methyl)-N-methyl-benzamido)ethyl acetate (Compound 36);
2-(3-(2-Methoxyethyl)-4-((1-(methylsulfonyl)piperidin-4-yl)methoxy)benzyl)-isoindoline (Compound 37);
4-(Isoindolin-2-ylmethyl)-7-((1-(methylsulfonyl)piperidin-4-yl)methoxy)-1H-indazole (Compound 38);
Methyl 4-((4-(isoindolin-2-ylmethyl)-2-nitrophenoxy)methyl)benzoate (Compound 39);
4-((4-(isoindolin-2-ylmethyl)-2-nitrophenoxy)methyl)-N,N-dimethylbenzamide (Compound 40);
4-(((5-(Isoindolin-2-ylmethyl)quinolin-8-yl)oxy)methyl)-N,N-dimethyl-benzamide (Compound 41);
4-((2-Acetyl-4-(isoindolin-2-ylmethyl)phenoxy)methyl)-N,N-dimethylbenzamide (Compound 42);
1-(2-((4-(Ethylsulfonyl)benzyl)oxy)-5-(isoindolin-2-ylmethyl)phenyl)ethan-1-one (Compound 43);
Imino(4-(((5-(isoindolin-2-ylmethyl)quinolin-8-yl)oxy)methyl)phenyl)(methyl)-λ⁶-sulfanone (Compound 44);
Imino(4-(((7-(isoindolin-2-ylmethyl)benzo[d]oxazol-4-yl)oxy)methyl)phenyl)-(methyl)-λ⁶-sulfanone (Compound 45);
4-(Isoindolin-2-ylmethyl)-7-((1-(methylsulfonyl)piperidin-4-yl)methoxy)-benzo[d]oxazole (Compound 46);
4-(Isoindolin-2-ylmethyl)-1-methyl-7-((1-(methylsulfonyl)piperidin-4-yl)-methoxy)-1H-indazole (Compound 47);
1-(5-(Isoindolin-2-ylmethyl)-2-((1-(methylsulfonyl)piperidin-4-yl)methoxy)-phenyl)ethan-1-one (Compound 48);
1-(4-((2-Acetyl-4-(isoindolin-2-ylmethyl)phenoxy)methyl)piperidin-1-yl)propan-1-one (Compound 49);
1-(5-(Isoindolin-2-ylmethyl)-2-((4-(methylsulfonyl)cyclohexyl)methoxy)-phenyl)ethan-1-one (Compound 50);
5-(Isoindolin-2-ylmethyl)-8-((1-(methylsulfonyl)piperidin-4-yl)methoxy)-quinoline (Compound 51);
4-((2-Amino-4-(isoindolin-2-ylmethyl)phenoxy)methyl)-N,N-dimethyl-benzamide (Compound 52);
4-((4-(Isoindolin-2-ylmethyl)-2-(methylsulfonamido)phenoxy)methyl)-N,N-dimethylbenzamide (Compound 53);
5-(Isoindolin-2-ylmethyl)-2-((4-(methylsulfonyl)benzyl)oxy)benzamide (Compound 54);
4-((4-(Isoindolin-2-ylmethyl)-2-(2,2,2-trifluoroacetamido)phenoxy)methyl)-N,N-dimethylbenzamide (Compound 55);
   and tautomers and pharmaceutically acceptable salts thereof.

Compounds of the invention may be administered to a patient in therapeutically effective amounts which range usually from about 0.5 to about 2000 mg, more typically form about 1 to about 500 mg, for example from about 2 to about 100 mg, daily depending on the age, sex, weight, ethnic group, condition of the patient, condition to be treated, administration route and the active ingredient used. The compounds of the invention can be formulated into dosage forms using the principles known in the art.

The compound can be given to a patient as such or in combination with suitable pharmaceutical excipients in the form of tablets, granules, capsules, suppositories, emulsions, suspensions or solutions. Choosing suitable ingredients for the composition is a routine for those of ordinary skill in the art. Suitable carriers, solvents, gel-forming ingredients, dispersion forming ingredients, antioxidants, colours, sweeteners, wetting compounds and other ingredients normally used in this field of technology may also be used. The compositions containing the active compound can be given enterally or parenterally, the oral route being the preferred way. The contents of the active compound in the composition is from about 0.5 to 100%, typically from about 0.5 to about 20%, per weight of the total composition.

The compounds of the invention can be given to the subject as the sole active ingredient or in combination with one of more other active ingredients for treatment of a particular disease.

In the treatment of a steroid receptor dependent disease or condition, such as endocrine cancers and disorders including prostate cancer and breast cancer, a combination of therapeutic agents and/or other treatments (e.g., radiation therapy) is often advantageous. The second (or third) agent to be administered may have the same or different mechanism of action than the primary therapeutic agent.

Accordingly, a compound of the invention may be administered in combination with other anti-cancer treatments useful in the treatment of cancers such as prostate cancer or breast cancer. For example, a compound of the invention can be packaged together with instructions that the compound is to be used in combination with other anti-cancer agents and treatments for the treatment of cancer. The present invention further comprises combinations of a compound of the invention and one or more additional agents in kit form, for example, where they are packaged together or placed in separate packages to be sold together as a kit, or where they are packaged to be formulated together.

According to one embodiment of the invention, the therapeutically effective amount of a compound of formula (I) or a pharmaceutically acceptable salt thereof is co-administered with a glucocorticoid and/or a mineralocorticoid and, optionally, with one or more anti-cancer agents.

Examples of suitable glucocorticoids include, but are not limited to, hydrocortisone, prednisone, prednisolone, methylprednisolone and dexamethasone.

Examples of suitable mineralocorticoids include, but are not limited to, fludrocortisone, deoxycorticosterone, 11-desoxycortisone and deoxycorticosterone acetate.

The optional other anti-cancer agents which can be administered in addition to a compound of formula (I) or a pharmaceutically acceptable salt thereof include, but are not limited to,
non-steroidal androgen receptor antagonists (e.g. enzalutamide, apalutamide and darolutamide);
steroidogenesis inhibitors (e.g. CYP17A1 inhibitors such as abiraterone acetate and seviteronel);
chemotherapeutic agents (e.g. docetaxel and paclitaxel);
antiestrogens (e.g. tamoxifen and fulvestrant);
epigenetic modulators (e.g. BET inhibitors and HDAC inhibitors);
mTOR inhibitors (e.g. everolimus);
AKT inhibitors (e.g. AZ5363);
radiopharmaceuticals (e.g. alpharadin);
GnRH/LHRH analogues (such as leuprorelin);
PI3K inhibitors (e.g. idelalisib); and
CDK4/6 inhibitors (e.g. ribocyclib).

According to one embodiment of the invention, the therapeutically effective amount of a compound of formula (I) or a pharmaceutically acceptable salt thereof is administered to a subject in need thereof in addition to a therapeutically effective amount of one or more anti-cancer agents selected from the list consisting of
non-steroidal androgen receptor antagonists (e.g. enzalutamide, apalutamide and darolutamide);
steroidogenesis inhibitors (e.g. CYP17A1 inhibitors such as abiraterone acetate and seviteronel);
chemotherapeutic agents (e.g. docetaxel and paclitaxel);
antiestrogens (e.g. tamoxifen and fulvestrant);
epigenetic modulators (e.g. BET inhibitors and HDAC inhibitors);
mTOR inhibitors (e.g. everolimus);
AKT inhibitors (e.g. AZ5363);
radiopharmaceuticals (e.g. alpharadin);
GnRH/LHRH analogues (such as leuprorelin);
PI3K inhibitors (e.g. idelalisib); and
CDK4/6 inhibitors (e.g. ribocyclib).

According to one embodiment of the invention, the therapeutically effective amount of a compound of formula (I) or a pharmaceutically acceptable salt thereof is administered to a subject in need thereof in addition to a therapeutically effective amount of a steroidogenesis inhibitor (e.g. a CYP17A1 inhibitor). Examples of suitable CYP17A1 inhibitors include, but are not limited to, abiraterone acetate and seviteronel.

According to another embodiment of the invention, the therapeutically effective amount of a compound of formula (I) or a pharmaceutically acceptable salt thereof is administered to a subject in need thereof in addition to a therapeutically effective amount of a non-steroidal androgen receptor antagonist. Examples of suitable non-steroidal androgen receptor (AR) antagonists include, but are not limited to, enzalutamide, apalutamide and darolutamide.

According to still another embodiment, the present invention provides a pharmaceutical combination comprising a compound of formula (I) or a pharmaceutically acceptable salt thereof and at least one additional active ingredient selected from the list consisting of
a glucocorticoid,
a mineralocorticoid,
a steroidogenesis inhibitor (e.g. a CYP17A1 inhibitor),
a non-steroidal androgen receptor antagonist,
chemotherapeutic agents (e.g. docetaxel and paclitaxel),
antiestrogens (e.g. tamoxifen and fulvestrant),
epigenetic modulators (e.g. BET inhibitors and HDAC inhibitors),
mTOR inhibitors (e.g. everolimus);
AKT inhibitors (e.g. AZ5363);
radiopharmaceuticals (e.g. alpharadin);
GnRH/LHRH analogues (such as leuprorelin);
PI3K inhibitors (e.g. idelalisib); and
CDK4/6 inhibitors (e.g. ribocyclib)
   for simultaneous, separate or sequential administration.

The above other therapeutic agents, when employed in combination with a compound of the invention can be used, for example, in those amounts indicated in the Physicians' Desk Reference (PDR) or as otherwise determined by one of ordinary skill in the art.

The compounds of the invention can be prepared by a variety of synthetic routes analogously to the methods known in the literature using suitable starting materials. The present invention will be explained in more detail by the following experiments and examples. The experiments and examples are meant only for illustrating purposes and do not limit the scope of the invention defined in claims.

### Examples

### Intermediate 1: Methyl 4-((4-formyl-2-methoxyphenoxy)methyl)benzoate

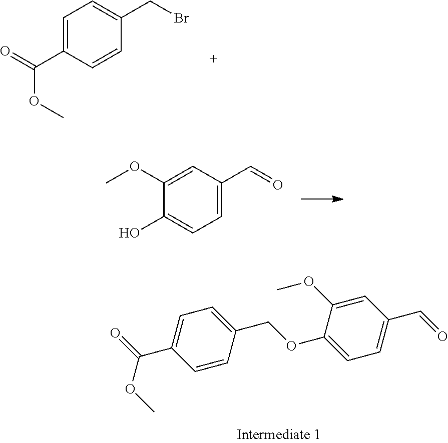 [SMILES: COC(=O)c1ccc(CBr)cc1 ; COc1cc(C=O)ccc1O ; COC(=O)c1ccc(COc2ccc(C=O)cc2OC)cc1]

To a stirring solution of vanillin (1.0 g, 6.57 mmol) in DMF (5 ml) were added K₂CO₃ (1.45 g, 10.52 mmol) and KI (0.11 g, 0.66 mmol). The reaction mixture was heated at 90° C. for 30 min. Methyl 4-(bromomethyl) benzoate (1.97 g, 8.54 mmol) was added. The reaction mixture was heated at 90° C. for 2 h. Water (10 ml) was added to the reaction mixture. The obtained precipitate was filtered, washed with water, and dried.

The crude product was purified by column chromatography to afford the title compound. ¹H NMR (400 MHz, DMSO-d₆) δ ppm: 3.86 (s, 6H), 5.33 (s, 2H), 7.25 (d, J=8.28 Hz, 1H), 7.44 (d, J=1.82 Hz, 1H), 7.52-7.65 (m, 3H), 7.91-8.08 (m, 2H), 9.85 (s, 1H). LC-MS: m/z 301.1 (M+H)⁺.

The following intermediates were prepared according to the procedure described for Intermediate 1 from the starting materials indicated on the table.

| No. | Structure | LC-MS | Starting material |
|---|---|---|---|
| Int-2 | 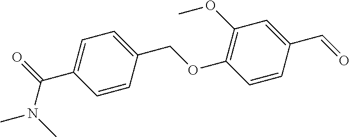 [SMILES: COc1cc(C=O)ccc1OCc1ccc(C(=O)N(C)C)cc1] | m/z 314.3 [M + H]⁺ | 4-(Chloromethyl)-N,N-dimethyl benzamide and vanillin |
| Int-3 | 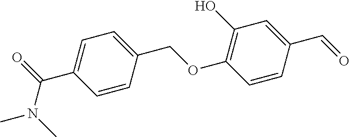 [SMILES: CN(C)C(=O)c1ccc(COc2ccc(C=O)cc2O)cc1] | m/z 300.3 [M + H]⁺ | 4-(Chloromethyl)-N,N-dimethyl benzamide and 3,4-Dihydro- benzaldehyde |
| Int-4 | 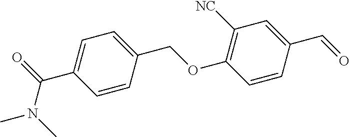 [SMILES: CN(C)C(=O)c1ccc(COc2ccc(C=O)cc2C#N)cc1] | m/z 309.3 [M + H]⁺ | 4-(Chloromethyl)-N,N-dimethyl benzamide and 5-Formyl-2- hydroxybenzonitrile |
| Int-5 | 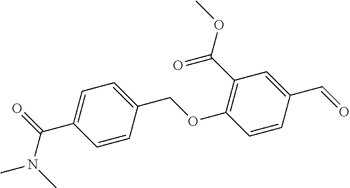 [SMILES: COC(=O)c1cc(C=O)ccc1OCc1ccc(C(=O)N(C)C)cc1] | m/z 342.3 [M + H]⁺ | 5-Formyl-2-hydroxybenzoic acid methyl ester and 4-(Chloro- methyl)-N,N-dimethyl benzamide |
| Int-6 | 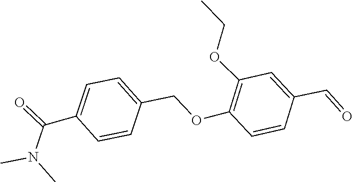 [SMILES: CCOc1cc(C=O)ccc1OCc1ccc(C(=O)N(C)C)cc1] | m/z 328.3 [M + H]⁺ | 4-(Chloromethyl)-N,N-dimethyl benzamide and 3-Ethoxy-4- hydroxybenzaldehyde |
| Int-7 | 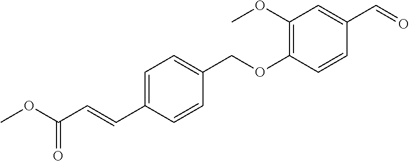 [SMILES: COC(=O)/C=C/c1ccc(COc2ccc(C=O)cc2OC)cc1] | m/z 327.3 [M + H]⁺ | Methyl-p-bromomethyl- cinnamate and vanillin |
| Int-8 | 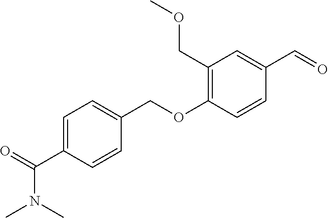 [SMILES: COCc1cc(C=O)ccc1OCc1ccc(C(=O)N(C)C)cc1] | m/z 328.3 [M + H]⁺ | 4-Hydroxy-3-(methoxymethyl)- benzaldehyde and 4-(Chloro- methyl)-N,N-dimethyl benzamide |
| Int-9 | 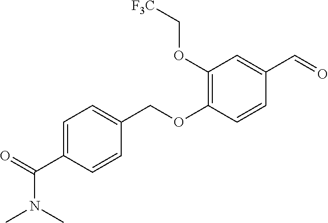 [SMILES: CN(C)C(=O)c1ccc(COc2ccc(C=O)cc2OCC(F)(F)F)cc1] | m/z 382.3 [M + H]⁺ | 4-((4-Formyl-2- hydroxyphenoxy)methyl)-N,N- dimethylbenzamide and 2-Iodo- 1,1,1-trifluoroethane |
| Int-10 | 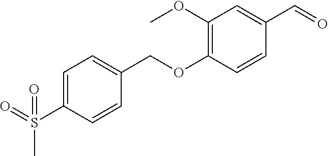 | m/z 368.5 [M + H]⁺ | 4-Methylsulfonylbenzyl bromide and vanillin |
| Int-11 | 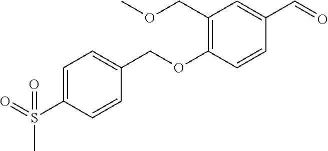 [SMILES: COCc1cc(C=O)ccc1OCc1ccc(S(C)(=O)=O)cc1] | m/z 335.2 [M + H]⁺ | 4-Hydroxy-3-(methoxymethyl)- benzaldehyde and 4-Methyl- sulfonylbenzyl bromide |
| Int-12 | 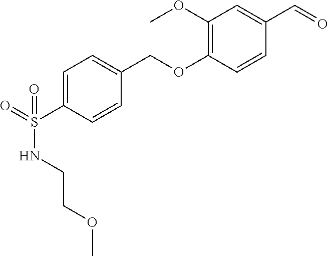 [SMILES: COCCNS(=O)(=O)c1ccc(COc2ccc(C=O)cc2OC)cc1] | m/z 380.3 [M + H]⁺ | 4-(Bromomethyl)-N-(2-methoxy- ethyl)benzenesulfonamide and vanillin |
| Int-13 | 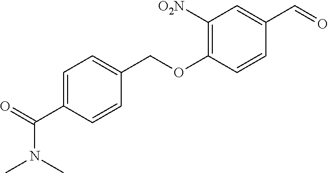 [SMILES: CN(C)C(=O)c1ccc(COc2ccc(C=O)cc2[N+](=O)[O-])cc1] | m/z 329.7 [M + H]⁺ | 4-(Chloromethyl)-N,N-dimethyl benzamide and 4-Hydroxy-3- nitrobenzaldehyde |
| Int-14 | 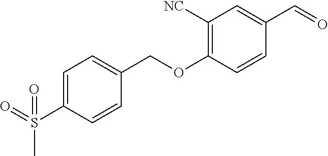 [SMILES: CS(=O)(=O)c1ccc(COc2ccc(C=O)cc2C#N)cc1] | m/z 317.3 [M + H]⁺ | 4-Methylsulfonylbenzyl bromide and 5-Formyl-2-hydroxy- benzonitrile |
| Int-15 | 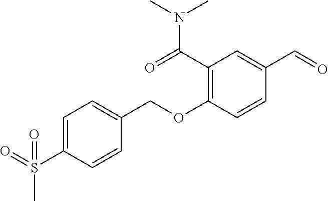 [SMILES: CN(C)C(=O)c1cc(C=O)ccc1OCc1ccc(S(C)(=O)=O)cc1] | m/z 362.3 [M + H]⁺ | 5-Formyl-2-hydroxy-N,N-di- methylbenzamide and 4-Methyl- sulfonylbenzyl bromide |
| Int-16 | 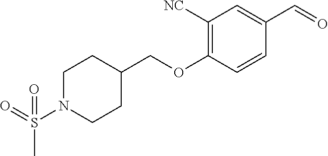 [SMILES: CS(=O)(=O)N1CCC(COc2ccc(C=O)cc2C#N)CC1] | m/z 323.4 [M + H]⁺ | 4-(Methanesulphonyloxymethyl)- 1-methanesulphonylpiperidine and 5-Formyl-2-hydroxy- benzonitrile |
| Int-17 | 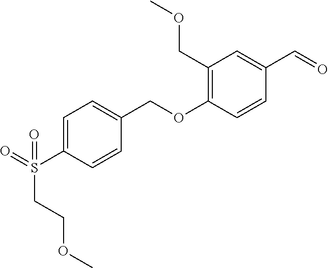 [SMILES: COCCS(=O)(=O)c1ccc(COc2ccc(C=O)cc2COC)cc1] | m/z 379.5 [M + H]⁺ | 4-Hydroxy-3-(methoxymethyl)- benzaldehyde and 1-(Bromo- methyl)-4-((2-methoxyethyl)- sulfonyl)benzene |
| Int-18 | 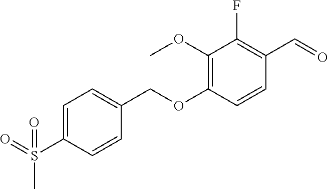 [SMILES: COc1c(OCc2ccc(S(C)(=O)=O)cc2)ccc(C=O)c1F] | m/z 339.3 [M + H]⁺ | 2-Fluoro-4-hydroxy-3-methoxy- benzaldehyde and 4-Methyl- sulfonylbenzyl bromide |
| Int-19 | 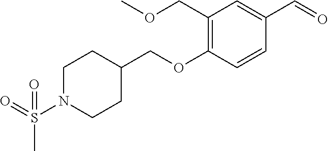 [SMILES: COCc1cc(C=O)ccc1OCC1CCN(S(C)(=O)=O)CC1] | m/z 342.2 [M + H]⁺ | (1-(Methylsulfonyl)piperidin-4- yl)methyl methanesulfonate and 4-Hydroxy-3-(methoxymethyl)- benzaldehyde |
| Int-20 | 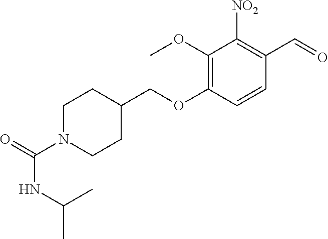 [SMILES: COc1c(OCC2CCN(C(=O)NC(C)C)CC2)ccc(C=O)c1[N+](=O)[O-]] | m/z 380.5 [M + H]⁺ | (1-(Isopropylcarbamoyl)piperidin- 4-yl)methyl 4-methylbenzene- sulfonate and 3-Hydroxy-4- methoxy-5-nitro-benzaldehyde |
| Int-21 | 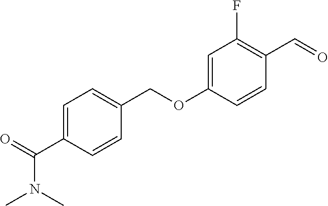 [SMILES: CN(C)C(=O)c1ccc(COc2ccc(C=O)c(F)c2)cc1] | m/z 302.4 [M + H]⁺ | 2-Fluoro-4-hydroxy benzaldehyde and 4-(Chloromethyl)-N,N-di- methylbenzamide |
| Int-22 | 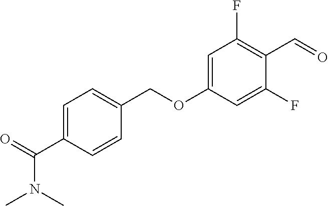 [SMILES: CN(C)C(=O)c1ccc(COc2cc(F)c(C=O)c(F)c2)cc1] | m/z 320.4 [M + H]⁺ | 2,6-Difluoro-4-hydroxy- benzaldehyde and 4-(Chloro- methyl)-N,N-dimethylbenzamide |
| Int-23 | 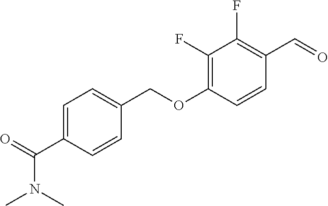 [SMILES: CN(C)C(=O)c1ccc(COc2ccc(C=O)c(F)c2F)cc1] | m/z 320.4 [M + H]⁺ | 2,3-Difluoro-4-hydroxy- benzaldehyde and 4-(Chloro- methyl)-N,N-dimethylbenzamide |
| Int-24 | 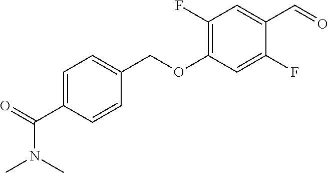 [SMILES: CN(C)C(=O)c1ccc(COc2cc(F)c(C=O)cc2F)cc1] | m/z 320.4 [M + H]⁺ | 2,5-Difluoro-4-hydroxy- benzaldehyde and 4-(Chloro- methyl)-N,N-dimethylbenzamide |
| Int-25 | 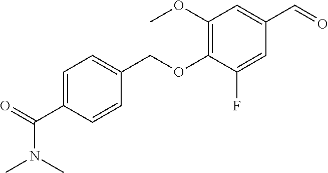 [SMILES: COc1cc(C=O)cc(F)c1OCc1ccc(C(=O)N(C)C)cc1] | m/z 332.4 [M + H]⁺ | 3-Fluoro-4-hydroxy-5-methoxy- benzaldehyde and 4-(Chloro- methyl)-N,N-dimethylbenzamide |
| Int-26 | 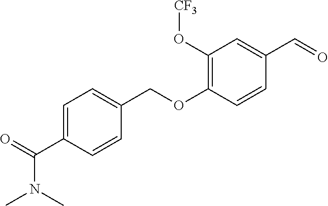 [SMILES: CN(C)C(=O)c1ccc(COc2ccc(C=O)cc2OC(F)(F)F)cc1] | m/z 368.3 [M + H]⁺ | 4-Hydroxy-3-(trifluoromethoxy)- benzaldehyde and 4-(Chloro- methyl)-N,N-dimethylbenzamide |
| Int-27 | 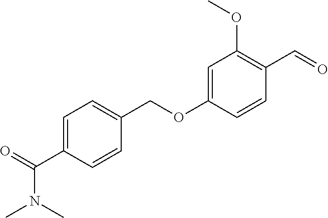 [SMILES: COc1cc(OCc2ccc(C(=O)N(C)C)cc2)ccc1C=O] | m/z 314.5 [M + H]⁺ | 4-Hydroxy-2-methoxy- benzaldehyde and 4-(Chloro- methyl)-N,N-dimethylbenzamide |
| Int-28 | 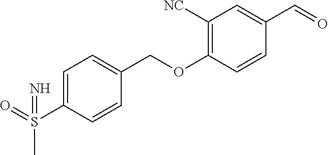 [SMILES: CS(=N)(=O)c1ccc(COc2ccc(C=O)cc2C#N)cc1] | m/z 315.5 [M + H]⁺ | 1-(Chloromethyl)-4-(S-methyl- sulfonimidoyl)benzene and 5- Formyl-2-hydroxybenzonitrile |
| Int-29 | 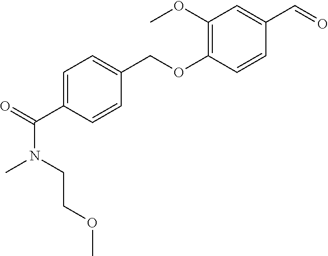 [SMILES: COCCN(C)C(=O)c1ccc(COc2ccc(C=O)cc2OC)cc1] | m/z 358.6 [M + H]⁺ | 4-(Chloromethyl)-N-(2-methoxy- ethyl)-N-methylbenzamide and Vanillin |
| Int-30 | 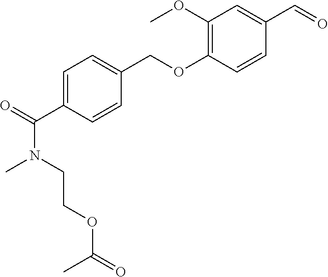 | m/z 358.6 [M + H]⁺ | 2-(4-(Chloromethyl)-N-methyl- benzamido)ethyl acetate and Vanillin |
| Int-31 | 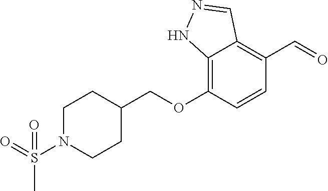 [SMILES: CS(=O)(=O)N1CCC(COc2ccc(C=O)c3cn[nH]c23)CC1] | m/z 338.2 [M + H]⁺ | 7-Hydroxy-1H-indazole-4- carbaldehyde and 4-(Methane- sulphonyloxymethyl)-1-methane- sulphonylpiperidine |
| Int-32 | 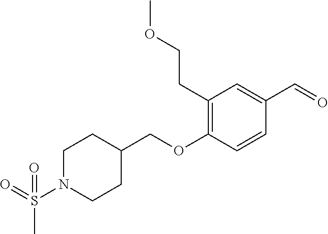 [SMILES: COCCc1cc(C=O)ccc1OCC1CCN(S(C)(=O)=O)CC1] | m/z 356.6 [M + H]⁺ | 4-(Methanesulphonyloxymethyl)- 1-methanesulphonylpiperidine and 4-Hydroxy-3-(2-methoxy- ethyl)benzaldehyde |

### Intermediate 33

### 4-((2-(Cyanomethoxy)-4-formylphenoxy)methyl)-N,N-dimethylbenzamide

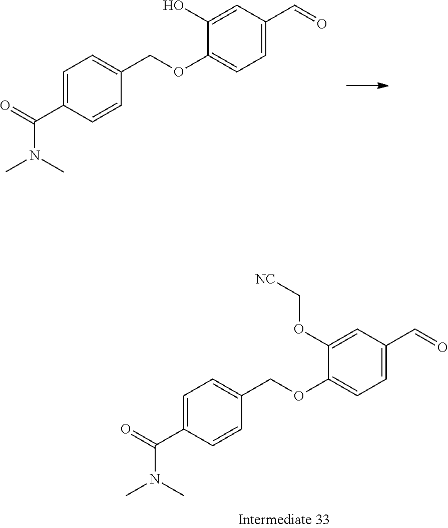 [SMILES: CN(C)C(=O)c1ccc(COc2ccc(C=O)cc2O)cc1 ; CN(C)C(=O)c1ccc(COc2ccc(C=O)cc2OCC#N)cc1]

To a stirring solution of 4-((4-formyl-2-hydroxyphenoxy)methyl)-N,N-dimethyl-benzamide (0.20 g, 0.67 mmol) in DMF (5 ml) were added K₂CO₃ (0.2 g, 1.33 mmol). The reaction mixture was heated at 100° C. for 30 min. Bromo acetonitrile (0.07 ml, 1.00 mmol) was added. The reaction mixture was heated at 120° C. for 1 h. Water (10 ml) was added to the reaction mixture. The product was extracted with EtOAc (10 ml), washed with water, dried with Na₂SO₄ and filtered. The obtained filtrate was evaporated and purified by column chromatography to afford 0.2 g of the title compound. LC-MS: m/z 339.3 (M+H)⁺.

### Example 1

### Methyl 4-((4-(isoindolin-2-ylmethyl)-2-methoxyphenoxy)methyl) benzoate (Compound 1)

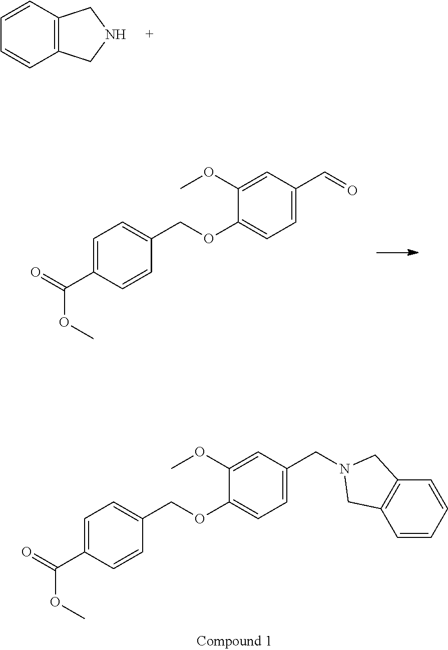 [SMILES: c1ccc2c(c1)CNC2 ; COC(=O)c1ccc(COc2ccc(C=O)cc2OC)cc1 ; COC(=O)c1ccc(COc2ccc(CN3Cc4ccccc4C3)cc2OC)cc1]

To a stirring solution of isoindoline (0.19 ml, 1.66 mmol) and glacial acetic acid (0.2 ml, 3.33 mmol) in DMF (5 ml) was added methyl 4-((4-formyl-2-methoxyphenoxy) methyl)benzoate (0.50 g, 1.66 mmol). After 30 min, the reaction mixture was cooled to 0° C. followed by addition of sodium triacetoxy borohydride (0.53 g, 2.50 mmol). The mixture was stirred overnight at room temperature and quenched with water (10 ml). The product was extracted with EtOAc. The combined extracts were washed with water, dried with Na₂SO₄, filtered and evaporated. The crude product was purified by chromatography to afford the title compound. ¹H NMR (Chloroform-d) δ: 7.95 (d, 1H), 7.90 (br d, 1H), 7.19-7.26 (m, 4H), 7.07 (d, 1H), 4.13 (s, 4H), 4.03-4.07 (m, 4H), 3.88-3.94 (m, 2H), 3.21 (s, 3H), 2.81 (s, 3H), 2.70-2.80 (m, 2H), 2.03 (br m, 2H), 1.50-1.62 (in, 3H). LC-MS: m/z 479.4 (M+H)⁺.

The following compounds were prepared according to the procedures described for Compound 1. The compound number, characterization data and starting materials are indicated on the table.

| No | Structure and starting material | ¹H NMR/LC-MS |
|---|---|---|
| 2 | 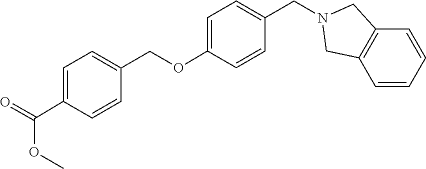 [SMILES: COC(=O)c1ccc(COc2ccc(CN3Cc4ccccc4C3)cc2)cc1] Starting materials: Methyl 4-((4- formylphenoxy)methyl)benzoate and Isoindoline | ¹H NMR (400 MHz, DMSO-d₆) δ: 3.79-3.90 (m, 3 H), 5.20 (s, 2 H), 7.00 (d, J = 7.60 Hz, 2 H), 7.13-7.26 (m, 4 H), 7.31 (d, J = 8.53 Hz, 2 H), 7.54-7.65 (m, 2 H), 7.90-8.06 (m, 2 H). LC-MS: m/z 374.4 (M + H)⁺. |
| 3 | 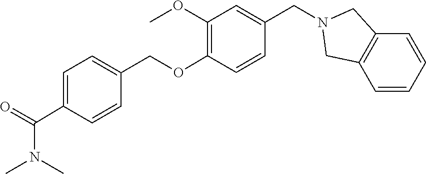 [SMILES: COc1cc(CN2Cc3ccccc3C2)ccc1OCc1ccc(C(=O)N(C)C)cc1] Starting materials: 4-((4-Formyl-2- methoxyphenoxy)methyl)-N,N- dimethylbenzamide and Isoindoline | ¹H NMR (400 MHz, DMSO-d₆) δ: 3.23 (br s, 6 H), 3.77 (s, 3 H), 3.86- 4.09 (m, 6 H), 5.11 (s, 2 H), 6.92 (d, J = 7.64 Hz, 1 H), 6.99-7.08 (m, 2 H), 7.18-7.28 (m, 4 H), 7.39-7.45 (m, 2 H), 7.46-7.53 (m, 2 H). LC-MS: m/z 417.4 (M + H)⁺. |
| 4 | 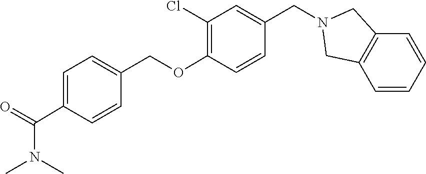 Starting materials: 4-((2-Chloro-4- formylphenoxy)methyl)-N,N-di- methylbenzamide and Isoindoline | ¹H NMR (400 MHz, Chloroform-d) δ: 3.07 (s, 6 H), 4.24-4.36 (m, 4 H), 4.86 (br dd, J = 14.30, 5.65 Hz, 2 H), 5.21 (s, 2 H), 7.04 (d, J = 8.53 Hz, 1 H), 7.25-7.38 (m, 4 H), 7.44-7.52 (m, 4 H), 7.60 (d, J = 1.95 Hz, 1 H), 7.81 (dd, J = 8.34, 1.69 Hz, 1 H), 13.39-13.56 (br s, 1 H). LCMS: m/z 521.4 (M + H)⁺. |
| 5 | 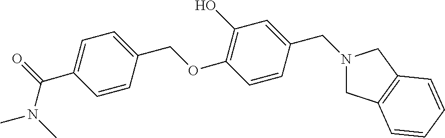 [SMILES: CN(C)C(=O)c1ccc(COc2ccc(CN3Cc4ccccc4C3)cc2O)cc1] Starting materials: 4-((4-Formyl-2- hydroxyphenoxy)methyl)-N,N- dimethylbenzamide and Isoindoline | ¹H NMR (400 MHz, Chloroform-d) δ: 3.00 (br s, 4 H), 3.12 (br s, 3 H), 3.82 (s, 2 H), 3.93 (s, 4 H), 5.13 (s, 2 H), 6.86 (s, 2 H), 7.17 (s, 4 H), 7.26 (s, 1 H), 7.45 (s, 4 H). LC-MS: m/z 403.4 (M + H)⁺. |
| 6 | 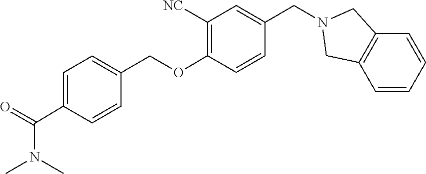 [SMILES: CN(C)C(=O)c1ccc(COc2ccc(CN3Cc4ccccc4C3)cc2C#N)cc1] Starting materials: 4-((2-Cyano-4- formylphenoxy)methyl)-N,N-di- methylbenzamide and Isoindoline | ¹H NMR (400 MHz, Chloroform-d) δ: 2.98 (br s, 3 H), 3.11 (br s, 3 H), 3.83 (s, 2 H), 3.89 (s, 4 H), 5.21 (s, 2 H), 6.96 (d, J = 8.72 Hz, 1 H), 7.18 (s, 4 H), 7.42-7.56 (m, 5 H), 7.63 (d, J = 2.13 Hz, 1 H). LCMS: m/z 47 (M + H)⁺. |
| 7 | 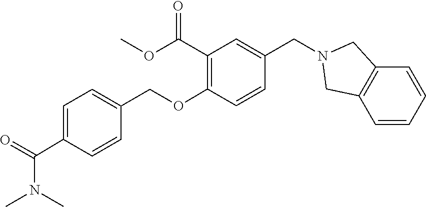 [SMILES: COC(=O)c1cc(CN2Cc3ccccc3C2)ccc1OCc1ccc(C(=O)N(C)C)cc1] Starting materials: Methyl 2-((4-(di- methylcarbamoyl)benzyl)oxy)-5- formylbenzoate and Isoindoline | ¹H NMR (400 MHz, Chloroform-d) δ: 2.99 (br s, 3 H), 3.12 (br s, 3 H), 3.85- 3.95 (m, 9 H), 5.21 (s, 2 H), 6.98 (d, J = 8.53 Hz, 1 H), 7.18 (s, 4 H), 7.45 (d, J = 8.20 Hz, 2 H), 7.42-7.51 (m , 1 H), 7.58 (d, J = 8.20 Hz, 2 H), 7.87 (d, J = 2.26 Hz, 1 H). LC-MS: m/z 445.4 (M + H)⁺. |
| 8 | 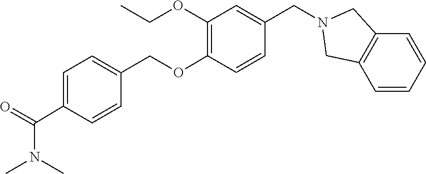 [SMILES: CCOc1cc(CN2Cc3ccccc3C2)ccc1OCc1ccc(C(=O)N(C)C)cc1] Starting materials: 4-((2-Ethoxy-4- formylphenoxy)methyl)-N,N-di- methylbenzamide and Isoindoline | ¹H NMR (400 MHz, Chloroform-d) δ: 1.45 (t, J = 7.00 Hz, 3 H), 2.98 (br s, 3 H), 3.10 (br s, 3 H), 3.82 (s, 2 H), 3.90 (s, 4 H), 4.12 (q, J = 6.96 Hz, 2 H), 5.15 (s, 2 H), 6.84 (d, J = 1.07 Hz, 2 H), 7.02 (s, 1 H), 7.23-7.31 (m, 4 H), 7.42 (d, J = 8.52 Hz 2 H), 7.49 (d, J = 8.52 Hz 2 H). LC-MS: m/z 431.5 (M + H)⁺. |
| 9 | 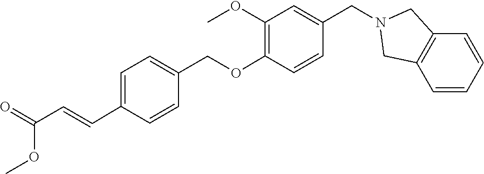 [SMILES: COC(=O)/C=C/c1ccc(COc2ccc(CN3Cc4ccccc4C3)cc2OC)cc1] Starting materials: (E)-Methyl 3-(4- ((4-formyl-2-methoxyphenoxy)- methyl)phenyl)acrylate and Isoindoline | ¹H NMR (400 MHz, DMSO-d₆) δ: 3.76 (d, J = 19.89 Hz, 6 H), 3.72-3.81 (m, 2 H), 3.82-4.07 (m, 4 H), 5.12 (s, 2 H), 6.66 (d, J = 16.12 Hz, 1 H), 6.88 (br d, J = 7.40 Hz, 1 H), 6.92-7.07 (m, 2 H), 7.22 (br d, J = 9.29 Hz, 4 H), 7.49 (d, J = 8.22 Hz, 2 H), 7.65-7.77 (m, 3 H). LC-MS: m/z 430.4 (M + H)⁺. |
| 10 | 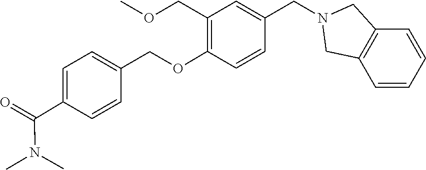 [SMILES: COCc1cc(CN2Cc3ccccc3C2)ccc1OCc1ccc(C(=O)N(C)C)cc1] Starting materials: 4-((4-Formyl-2- (methoxymethyl)phenoxy)methyl)- N,N-dimethylbenzamide and Isoindoline | ¹H NMR (400 MHz, Chloroform-d) δ: 2.99 (br s, 3 H), 3.11 (br s, 3 H), 3.45 (s, 3 H), 3.85 (s, 2 H), 3.91 (s, 4 H), 4.57 (s, 2 H), 5.12 (s, 2 H), 6.88 (d, J = 8.34 Hz, 1 H), 7.16 (s, 4 H), 7.24- 7.31 (m, 1 H), 7.42-7.48 (m, 5 H). LC-MS: m/z 431.4 (M + H)⁺. |
| 11 | 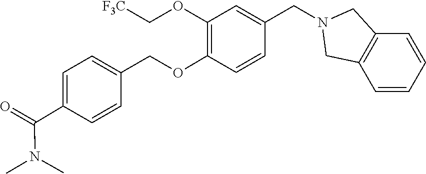 [SMILES: CN(C)C(=O)c1ccc(COc2ccc(CN3Cc4ccccc4C3)cc2OCC(F)(F)F)cc1] Starting materials: 4-((4-Formyl-2- (2,2,2-trifluoroethoxy)phenoxy)- methyl)-N,N-dimethylbenzamide and Isoindoline | ¹H NMR (400 MHz, Chloroform-d) δ: 2.99 (br s, 3 H), 3.12 (br s, 3 H), 3.83 (s, 2 H), 3.91 (s, 4 H), 4.42 (q, J = 8.34 Hz, 2 H), 5.15 (s, 2 H), 6.92 (d, J = 8.22 Hz, 1 H), 7.02 (dd, J = 8.22, 2.01 Hz, 1 H), 7.10 (d, J = 1.95 Hz, 1 H), 7.18 (s, 4 H), 7.42-7.51 (m, 4 H). LC-MS: m/z 485.5 (M + H)⁺. |
| 12 | 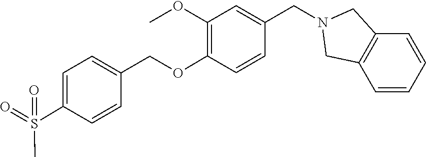 [SMILES: COc1cc(CN2Cc3ccccc3C2)ccc1OCc1ccc(S(C)(=O)=O)cc1] Starting materials: 3-Methoxy-4-((4- (methylsulfonyl)benzyl)oxy)- benzaldehyde and Isoindoline | ¹H NMR (400 MHz, Chloroform-d) δ: 3.07 (s, 3 H), 3.87 (s, 3 H), 4.02 (s, 3 H), 4.16-4.43 (m, 6 H), 5.26 (s, 2 H), 6.80 (br d, J = 6.59 Hz, 1 H), 6.90 (br d, J = 6.56 Hz, 1 H), 7.27-7.41 (m, 5 H), 7.65 (br d, J = 8.09 Hz, 3 H), 7.97 (d, J = 8.03 Hz, 2 H). LC-MS: m/z 424.4 (M + H)⁺. |
| 13 | 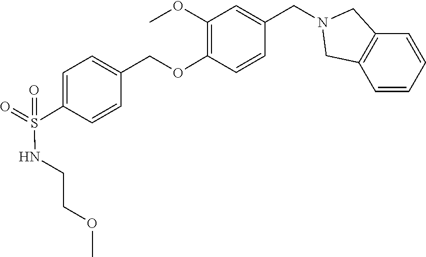 [SMILES: COCCNS(=O)(=O)c1ccc(COc2ccc(CN3Cc4ccccc4C3)cc2OC)cc1] Starting materials: 4-((4-Formyl-2- methoxyphenoxy)methyl)-N-(2- methoxyethyl)benzenesulfonamide and Isoindoline | ¹H NMR (400 MHz, Chloroform-d) δ: 3.10-3.15 (m, 2 H), 3.27 (s, 3 H), 3.41 (dd, J = 5.49, 4.61 Hz, 2 H), 3.85 (s, 2 H), 3.89-3.94 (m, 6 H), 4.80 (t, J = 5.99 Hz, 1 H), 5.21 (s, 2 H), 6.78- 6.87 (m, 2 H), 7.04 (s, 1 H), 7.18 (s, 4 H), 7.60 (d, J = 8.42 Hz, 2 H), 7.86 (d, J = 8.41 Hz, 2 H). LC-MS: m/z 483.4 (M + H)⁺. |
| 14 | 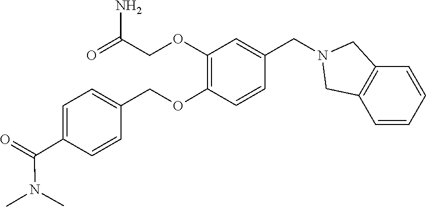 Starting materials: 4-((2-(Cyano- methoxy)-4-formylphenoxy)methyl)- N,N-dimethylbenzamide and Isoindoline | ¹H NMR (400 MHz, Chloroform-d) δ: 2.99 (br s, 3H), 3.12 (br s, 3 H), 4.01 (s, 2 H), 4.17 (s, 4 H), 4.60 (s, 2 H), 5.16 (s, 2 H), 6.88-7.09 (m, 3 H), 7.21-7.29 (m, 6 H), 7.41-7.51 (m, 4 H). LC-MS: m/z 483.7 (M + H)⁺. |
| 15 | 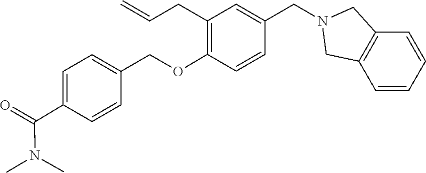 [SMILES: C=CCc1cc(CN2Cc3ccccc3C2)ccc1OCc1ccc(C(=O)N(C)C)cc1] Starting materials: 4-((2-Allyl-4- formylphenoxy)methyl)-N,N-di- methylbenzamide and Isoindoline | ¹H NMR (400 MHz, Chloroform-d) δ: 3.05 (br s, 3 H), 3.10 (br s, 3 H), 3.47 (br d, J = 5.90 Hz, 2 H), 4.52 (br s, 2 H), 4.62 (br s, 4 H), 4.95-5.13 (m, 2 H), 5.22 (s, 2 H), 5.95-6.09 (m, 1 H), 7.11 (br d, J = 7.65 Hz, 1 H), 7.35- 7.41 (m, 6 H), 7.45 (br d, J = 7.72 Hz, 2 H), 7.55 (br d, J = 7.78 Hz, 2 H). LC-MS: m/z 427.5 (M + H)⁺. |
| 16 | 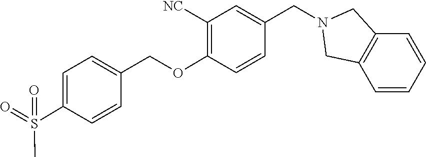 [SMILES: CS(=O)(=O)c1ccc(COc2ccc(CN3Cc4ccccc4C3)cc2C#N)cc1] Starting materials: 5-Formyl-2-((4- (methylsulfonyl)benzyl)oxy)benzo- nitrile and and Isoindoline | ¹H NMR (400 MHz, Chloroform-d) δ: 3.06-3.09 (m, 3 H), 3.87 (s, 2 H), 3.92 (s, 4 H), 5.29 (s, 2 H), 6.92-7.00 (m, 1 H), 7.17-7.22 (m, 4 H), 7.59 (br d, J = 8.66 Hz, 1 H), 7.67 (s, 1 H), 7.68- 7.72 (m, 2 H), 8.00 (d, J = 7.73 Hz, 2 H). LC-MS: m/z 419.4 (M + H)⁺. |
| 17 | 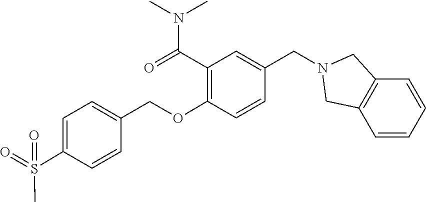 [SMILES: CN(C)C(=O)c1cc(CN2Cc3ccccc3C2)ccc1OCc1ccc(S(C)(=O)=O)cc1] Starting materials: 5-Formyl-N,N-di- methyl-2-((4-(methylsulfonyl)benzyl)- oxy)benzamide and Isoindoline | ¹H NMR (400 MHz, Chloroform-d) δ: 2.90 (s, 3 H), 3.07 (s, 3 H), 3.13 (s, 3 H), 3.89 (br s, 2 H), 3.92 (s, 4 H), 5.21 (s, 2 H), 6.89 (d, J = 8.41 Hz, 1 H), 7.16- 7.20 (m, 4 H), 7.34 (d, J = 2.13 Hz, 1 H), 7.37 (dd, J = 8.41, 2.20 Hz, 1 H), 7.61 (d, J = 8.53 Hz, 2 H), 7.93-7.97 (m, 2 H). LC-MS: m/z 465.5 (M + H)⁺. |
| 18 | 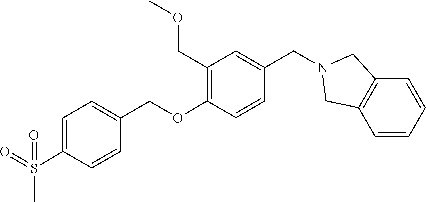 [SMILES: COCc1cc(CN2Cc3ccccc3C2)ccc1OCc1ccc(S(C)(=O)=O)cc1] Starting materials: 3-(Methoxy- methyl)-4-((4-(methylsulfonyl)- benzyl)oxy)benzaldehyde and Isoindoline | ¹H NMR (400 MHz, Chloroform-d) δ: 3.05 (s, 3 H), 3.45 (s, 3 H), 3.85 (s, 2 H), 3.90 (s, 4 H), 4.57 (s, 2 H), 5.18 (s, 2 H), 6.85 (d, J = 8.41 Hz, 1 H), 7.15 (s, 4 H), 7.22-7.36 (m, 1 H), 7.45 (d, J = 2.20 Hz, 1 H), 7.64 (d, J = 7.92 Hz, 2 H), 7.95 (d, J = 7.92 Hz, 2 H. LC-MS: m/z 438.4 (M + H)⁺. |
| 19 | 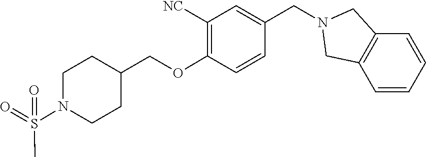 [SMILES: CS(=O)(=O)N1CCC(COc2ccc(CN3Cc4ccccc4C3)cc2C#N)CC1] Starting materials: 5-Formyl-2-((1- (methylsulfonyl)piperidin-4-yl)- methoxy)benzonitrile and Isoindoline | ¹H NMR (400 MHz, DMSO-d₆) δ: 1.40 (qd, J = 12.10, 4.11 Hz, 2 H), 1.83- 1.99 (m, 3 H), 2.76 (td, J = 11.95, 2.13 Hz, 2 H), 3.57-3.66 (m, 3 H), 3.60 (br s, 2 H), 3.63 (br s, 2 H), 3.95 (br s, 4 H), 4.03-4.11 (m, 2 H), 7.14-7.34 (m, 4 H), 7.66-7.80 (m, 3 H). LC-MS: m/z 426.5 (M + H)⁺. |
| 20 | 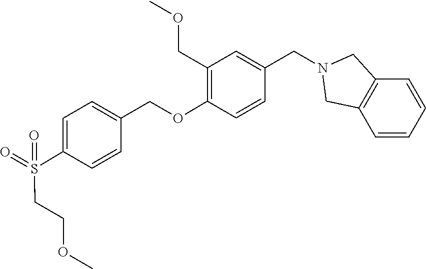 [SMILES: COCCS(=O)(=O)c1ccc(COc2ccc(CN3Cc4ccccc4C3)cc2COC)cc1] Starting materials: 4-((4-((2-Methoxy- ethyl)sulfonyl)benzyl)oxy)-3- (methoxymethyl)benzaldehyde and Isoindoline | ¹H NMR (400 MHz, Chloroform-d) δ: 3.24 (s, 3 H), 3.37-3.42 (m, 2 H), 3.46 (s, 3 H), 3.73-3.79 (m, 2 H), 3.86 (s, 2 H), 3.91 (s, 4 H), 4.57 (s, 2 H), 5.20 (s, 2 H), 6.85 (d, J = 8.34 Hz, 1 H), 7.17 (s, 4 H), 7.29 (dd, J-8.31, 2.16 Hz, 1 H), 7.43-7.47 (m, 1 H), 7.64 (d, J = 8.34 Hz, 2 H), 7.92-7.97 (m, 2 H). LC-MS: m/z 482.6 (M + H)⁺. |
| 21 | 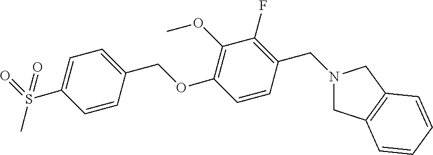 [SMILES: COc1c(OCc2ccc(S(C)(=O)=O)cc2)ccc(CN2Cc3ccccc3C2)c1F] Starting materials: 2-Fluoro-3- methoxy-4-((4-(methylsulfonyl)- benzyl)oxy)benzaldehyde and Isoindoline | ¹H NMR (Chloroform-d) δ: 7.95-8.00 (m, 2H), 7.66 (d, 2H), 7.17 (s, 4H), 7.07 (m, 1H), 6.68 (m, 1H), 5.22 (s, 2H), 3.97 (d, 3H), 3.96 (s, 4H), 3.91 (d, 2H), 3.06 (s, 3H). LC-MS: m/z 442.4 (M + H)⁺. |
| 22 | 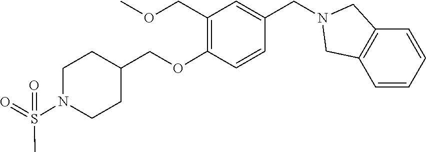 [SMILES: COCc1cc(CN2Cc3ccccc3C2)ccc1OCC1CCN(S(C)(=O)=O)CC1] Starting materials: 3-(Methoxy- methyl)-4-((1-(methylsulfonyl)- piperidin-4-yl)methoxy)benzaldehyde and Isoindoline | ¹H NMR (Chloroform-d) δ: 7.40 (d, 1H), 7.29 (m, 1H), 7.16 (s, 4H), 6.81 (d, 1H), 4.49 (s, 2H), 3.91 (s, 4H), 3.87 (d, 2H), 3.85 (s, 2H), 3.43 (s, 3H), 3.28 (br d, 2H), 2.80 (s, 2H), 2.73 (m, 2H), 1.93-2.01 (m, 2H), 1.60-1.65 (m, 1H), 1.48-1.60 (m, 2H). LC-MS: m/z 445.9 (M + H)⁺. |
| 23 | 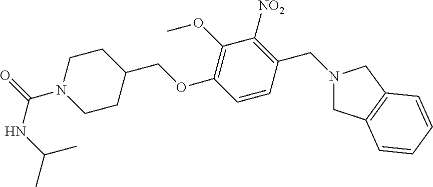 [SMILES: COc1c(OCC2CCN(C(=O)NC(C)C)CC2)ccc(CN2Cc3ccccc3C2)c1[N+](=O)[O-]] Starting materials: 4-((4-Formyl-2- methoxy-3-nitrophenoxy)methyl)-N- isopropylpiperidine-1-carboxamide and Isoindoline | ¹H NMR (Chloroform-d) δ: 7.23 (d, 1H), 7.13-7.19 (m, 4H), 6.96 (d, 1H),4.22 (br d, 1H), 3.96-4.03 (m, 2H), 3.96-4.03 (m, 1H), 3.93 (s, 3H), 3.90 (s, 4H), 3.89 (d, 2H), 3.85 (s, 2H), 2.82 (m, 2H), 2.00-2.11 (m, 1H), 1.87 (br d, 2H), 1.36 (qd, 2H), 1.16 (d, 6H). LC-MS: m/z 483.8 (M + H)⁺. |
| 24 | 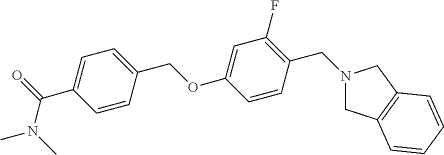 Starting materials: 4-((3-Fluoro-4- formylphenoxy)methyl)-N,N-di- methylbenzamide and Isoindoline | ¹H NMR (Chloroform-d) δ: 7.42-7.48 (m, 4H), 7.33-7.40 (m, 1H), 7.17 (s, 4H), 6.76 (m, 1H), 6.70 (m, 1H), 5.08 (s, 2H), 3.96 (s, 4H), 3.91 (s, 2H), 3.12 (br s, 3H), 2.99 (br s, 3H). LC-MS: m/z 363.4 (M + H)⁺. |
| 25 | 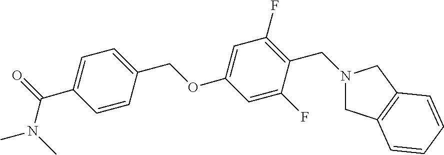 [SMILES: CN(C)C(=O)c1ccc(COc2cc(F)c(CN3Cc4ccccc4C3)c(F)c2)cc1] Starting materials: 4-((3,5-Difluoro-4- formylphenoxy)methyl)-N,N-di- methylbenzamide and Isoindoline | ¹H NMR (Chloroform-d) δ: 7.45 (d, 4H), 7.13-7.21 (m, 4H), 6.48-6.60 (m, 2H), 5.13 (s, 2H), 3.97 (s, 4H), 3.95 (s, 2H), 3.12 (br s, 3H), 2.99 (br s, 3H). LC-MS: m/z 422.5 (M + H)⁺. |
| 26 | 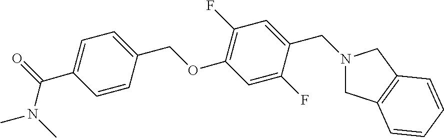 Starting materials: 4-((2,5-Difluoro-4- formylphenoxy)methyl)-N,N-di- methylbenzamide and Isoindoline | ¹H NMR (Chloroform-d) δ: 7.42-7.50 (m, 4H), 7.20-7.25 (m, 1H), 6.72 (m, 1H), 5.14 (s, 2H), 3.95 (s, 4H), 3.87 (d, 2H), 3.12 (br s, 3H), 2.99 (br s, 3H). LC-MS: m/z 320.3 (M + H)⁺. |
| 27 | 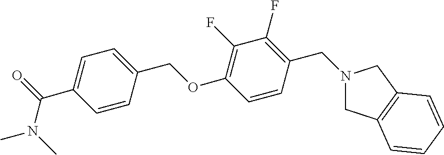 [SMILES: CN(C)C(=O)c1ccc(COc2ccc(CN3Cc4ccccc4C3)c(F)c2F)cc1] Starting materials: 4-((2,3-Difluoro-4- formylphenoxy)methyl)-N,N-di- methylbenzamide and Isoindoline | ¹H NMR (Chloroform-d) δ: 7.42-7.50 (m, 4H), 7.18 (s, 4H), 7.06-7.14 (m, 1H), 6.75 (m, 1H), 5.17 (s, 2H), 3.96 (s, 4H), 3.92 (d, 2H), 3.12 (br s, 3H), 2.99 (br s, 3H). LC-MS: m/z 423.4 (M + H)⁺. |
| 28 | 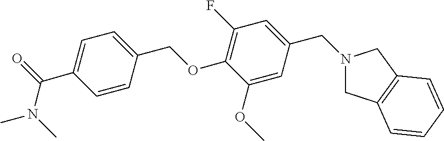 [SMILES: COc1cc(CN2Cc3ccccc3C2)cc(F)c1OCc1ccc(C(=O)N(C)C)cc1] Starting materials: 4-((2-Fluoro-4- formyl-6-methoxyphenoxy)methyl)- N,N-dimethylbenzamide and Isoindoline | ¹H NMR (Chloroform-d) δ: 7.39-7.54 (m, 4H), 7.20 (s, 4H), 6.81 (m, 1H), 6.76 (m, 1H), 5.10 (s, 2H), 3.96 (s, 4H), 3.86 (s, 3H), 3.84 (s, 2H), 3.11 (br s, 3H), 2.97 (br s, 3H). LC-MS: m/z 435.8 (M + H)⁺. |
| 29 | 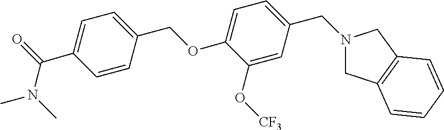 [SMILES: CN(C)C(=O)c1ccc(COc2ccc(CN3Cc4ccccc4C3)cc2OC(F)(F)F)cc1] Starting materials: 4-((4-Formyl-2-(tri- fluoromethoxy)phenoxy)methyl)-N,N- dimethylbenzamide and Isoindoline | ¹H NMR (Chloroform-d) δ: 7.42-7.49 (m, 5H), 7.34 (s, 1H), 7.19 (s, 4H), 6.97 (d, 1H), 5.17 (s, 2H), 3.96 (s, 4H), 3.88 (s, 2H), 3.12 (br s, 3H), 3.00 (br s, 3H). LC-MS: m/z 471.8 (M + H)⁺. |
| 30 | 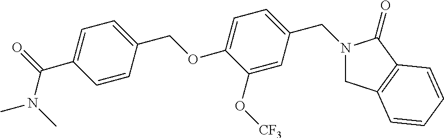 [SMILES: CN(C)C(=O)c1ccc(COc2ccc(CN3Cc4ccccc4C3=O)cc2OC(F)(F)F)cc1] (obtained as a side product) Starting materials: 4-((4-Formyl-2-(tri- fluoromethoxy)phenoxy)methyl)-N,N- dimethylbenzamide and Isoindoline | LC-MS: m/z 485.5 (M + H)⁺. |
| 31 | 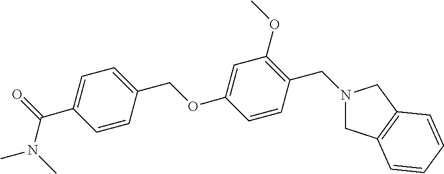 [SMILES: COc1cc(OCc2ccc(C(=O)N(C)C)cc2)ccc1CN1Cc2ccccc2C1] Starting materials: 4-((4-Formyl-3- methoxyphenoxy)methyl)-N,N-di- methylbenzamide and Isoindoline | ¹H NMR (Chloroform-d) δ: 7.42-7.49 (m, 4H), 7.31 (d, 1H), 7.16 (s, 4H), 6.51-6.57 (m, 2H), 5.08 (s, 2H), 4.00 (s, 4H), 3.91 (s, 2H), 3.81 (s, 3H), 3.11 (br s, 3H), 2.98 (br s, 3H). LC-MS: m/z 417.6 (M + H)⁺. |
| 32 | 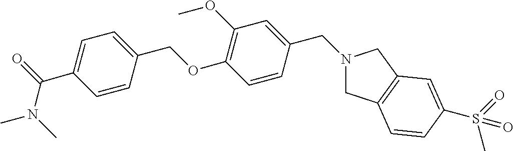 [SMILES: COc1cc(CN2Cc3ccc(S(C)(=O)=O)cc3C2)ccc1OCc1ccc(C(=O)N(C)C)cc1] Starting materials: 4-((4-Formyl-2- methoxyphenoxy)methyl)-N,N-di- methylbenzamide and 5-(Methyl- sulfonyl)isoindoline | ¹H NMR (Chloroform-d) δ: 7.79 (m, 1H), 7.76 (s, 1H), 7.41-7.51 (m, 4H), 7.36 (d, 1H), 6.98 (s, 1H), 6.83 (d, 2H), 5.18 (s, 2H), 3.97 (s, 4H), 3.91 (s, 3H), 3.85 (s, 2H), 3.11 (br s, 3H), 3.02 (s, 3H), 2.99 (br s, 2H). LC-MS: m/z 495.4 (M + H)⁺. |
| 33 | 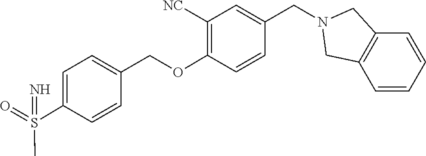 [SMILES: CS(=N)(=O)c1ccc(COc2ccc(CN3Cc4ccccc4C3)cc2C#N)cc1] Starting materials: 5-Formyl-2-((4-(S- methylsulfonimidoyl)benzyl)oxy)- benzonitrile and Isoindoline | ¹H NMR (Chloroform-d) δ: 8.02-8.08 (m, 2H), 7.64-7.69 (m, 3H), 7.58 (m, 1H), 7.28-7.32 (m, 1H), 7.15-7.23 (m, 4H), 6.97 (d, 1H), 5.29 (s, 2H), 3.91 (s, 4H), 3.86 (s, 2H), 3.13 (s, 3H). LC-MS: m/z 418.2 (M + H)⁺. |
| 34 | 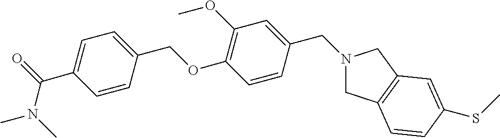 Starting materials: 4-((4-Formyl-2- methoxyphenoxy)methyl)-N,N-di- methylbenzamide and 5-(Methylthio)- isoindoline HCl | ¹H NMR (Chloroform-d) δ: 7.40-7.51 (m, 4H), 7.09-7.14 (m, 3H), 7.01 (d, 1H), 6.81-6.84 (m, 2H), 5.17 (s, 2H), 3.90 (s, 3H), 3.89 (br d, 4H), 3.83 (s, 2H), 3.11 (br s, 3H), 2.99 (br s, 3H), 2.46 (s, 3H). LC-MS: m/z 463.5 (M + H)⁺. |
| 35 | 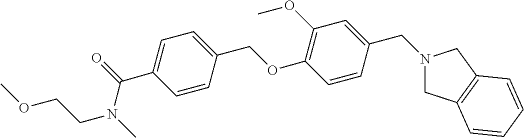 [SMILES: COCCN(C)C(=O)c1ccc(COc2ccc(CN3Cc4ccccc4C3)cc2OC)cc1] Starting materials: 4-((4-Formyl-2- methoxyphenoxy)methyl)-N-(2- methoxyethyl)-N-methylbenzamide and Isoindoline | ¹H NMR (Chloroform-d) δ: 7.40-7.50 (m, 4H), 7.18 (s, 4H), 7.02 (d, 1H), 6.82-6.85 (m, 2H), 5.17 (s, 2H), 3.92 (s, 4H), 3.90 (s, 3H), 3.84 (s, 2H), 3.63-3.75 (m, 3H), 3.44 (br s, 2H), 3.28 (br s, 2H), 3.02-3.15 (m, 3H). LC-MS: m/z 461.9 (M + H)⁺. |
| 36 | 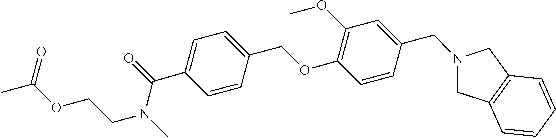 [SMILES: COc1cc(CN2Cc3ccccc3C2)ccc1OCc1ccc(C(=O)N(C)CCOC(C)=O)cc1] Starting materials: 2-(4-((4-Formyl-2- methoxyphenoxy)methyl)-N-methyl- benzamido)ethyl acetate and Isoindoline | ¹H NMR (Chloroform-d) δ: 7.47-7.52 (m, 2H), 7.39-7.43 (m, 2H), 7.18 (s, 4H), 7.02 (d, 1H), 6.81-6.85 (m, 2H), 5.17 (s, 2H), 4.06-4.22 (m, 2H), 3.91 (d, 6H), 3.76-3.88 (m, 3H), 3.12 (br s, 2H), 3.03 (br s, 3H), 2.08 (br s, 3H). LC-MS: m/z 489.8 (M + H)⁺. |
| 37 | 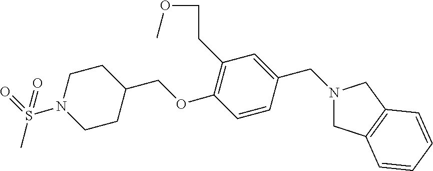 [SMILES: COCCc1cc(CN2Cc3ccccc3C2)ccc1OCC1CCN(S(C)(=O)=O)CC1] Starting materials: 3-(2-Methoxy- ethyl)-4-((1- (methylsulfonyl)piperidin-4- yl)methoxy)benzaldehyde and Isoindoline | ¹H NMR (Chloroform-d) δ: 7.28-7.33 (m, 3H), 7.22-7.26 (m, 2H), 6.83 (d, 2H), 4.40 (s, 4H), 4.18 (s, 2H), 3.85- 3.92 (m, 4H), 3.58 (m, 2H), 3.35 (s, 3H), 2.90 (m, 2H), 2.81 (s, 3H), 2.73 (m, 2H), 1.94-2.00 (m, 3H), 1.48-1.60 (m, 2H). LC-MS: m/z 459.9 (M + H)⁺. |
| 38 | 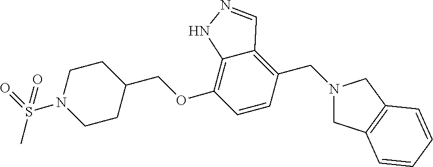 [SMILES: CS(=O)(=O)N1CCC(COc2ccc(CN3Cc4ccccc4C3)c3cn[nH]c23)CC1] Starting materials: 7-((1-(Methyl- sulfonyl)piperidin-4-yl)methoxy)-3H- indazole-4-carbaldehyde and Isoindoline HCl | ¹H NMR (Chloroform-d) δ: 8.27 (s, 1H), 7.14-7.19 (m, 4H), 7.06 (d, 1H), 6.67 (d, 1H), 4.15 (s, 2H), 4.04 (d, 2H), 3.96 (s, 4H), 3.88-3.94 (m, 2H), 2.82 (s, 3H), 2.70-2.78 (m, 2H), 1.99- 2.08 (m, 3H), 1.51-1.66 (m, 8H). LC-MS: m/z 441.5 (M + H)⁺. |

### Example 2

### Methyl 4-((4-(isoindolin-2-ylmethyl)-2-nitrophenoxy)methyl)benzoate (Compound 39)

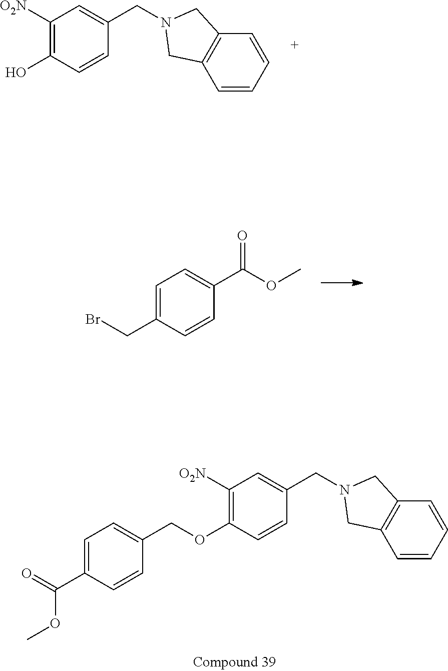 [SMILES: O=[N+]([O-])c1cc(CN2Cc3ccccc3C2)ccc1O ; COC(=O)c1ccc(CBr)cc1 ; COC(=O)c1ccc(COc2ccc(CN3Cc4ccccc4C3)cc2[N+](=O)[O-])cc1]

To a solution of 4-(isoindolin-2-ylmethyl)-2-nitrophenol (0.10 g, 0.37 mmol) in DMSO (2 ml) were added K₂CO₃ (83.0 mg, 0.60 mmol). The reaction mixture was stirred at 80° C. for 30 min. Methyl 4-(bromomethyl)benzoate (0.11 g, 0.49 mmol) was added. The reaction mixture was heated at 80° C. for 3 h. Water (10 ml) was added, and the product was extracted with EtOAc. The combined extracts were washed with water, dried with Na₂SO₄ filtered and evaporated. The crude product was purified by chromatography to afford 14.0 mg of the title compound. ¹H NMR (400 MHz, Chloroform-d) δ: 3.89 (s, 2H), 3.92-3.97 (m, 4H), 5.29 (s, 2H), 7.07 (d, J=8.60 Hz, 1H), 7.19 (s, 4H), 7.36-7.59 (m, 3H), 7.93 (d, J=2.20 Hz, 1H), 8.06-8.14 (n, 2H). LC-MS: m/z 419.3 (M+H)⁺.

The following compounds were prepared according to the procedures described for Compound 39. The compound number, characterization data and starting materials are indicated on the table.

| No | Structure and starting material | 1H NMR/LC-MS |
|---|---|---|
| 40 | 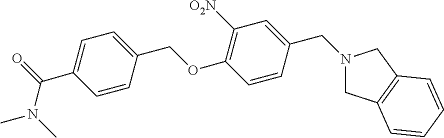 [SMILES: CN(C)C(=O)c1ccc(COc2ccc(CN3Cc4ccccc4C3)cc2[N+](=O)[O-])cc1] Starting materials: 4-(Isoindolin-2-yl- methyl)-2-nitrophenol and 4-(Chloro- methyl)-N,N-dimethylbenzamide | ¹H NMR (400 MHz, DMSO-d₆) δ: 2.88-3.02 (m, 6 H), 3.85 (s, 4 H), 3.88 (s, 2 H), 5.36 (s, 2 H), 7.15-7.26 (m, 4 H), 7.42-7.54 (m, 5 H), 7.67 (dd, J = 8.63, 2.16 Hz, 1 H), 7.89 (d, J = 2.13 Hz, 1 H). LC-MS: m/z 432.6 (M + H)⁺. |
| 41 | 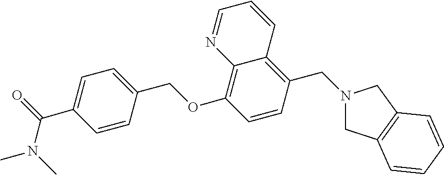 [SMILES: CN(C)C(=O)c1ccc(COc2ccc(CN3Cc4ccccc4C3)c3cccnc23)cc1] Starting materials: 5-(Isoindolin-2-yl- methyl)quinolin-8-ol and 4-(Chloro- methyl)-N,N-dimethylbenzamide | ¹H NMR (Chloroform-d) δ: 9.00 (m, 1H), 8.63 (m, 1H), 7.42-7.59 (m, 6H), 7.17-7.26 (m, 4H), 7.00 (d, 1H), 5.44 (s, 2H), 4.45 (s, 2H), 4.21 (s, 4H), 3.11 (br s, 3H), 2.99 (s, 3H). LC-MS: m/z 438.4 (M + H)⁺. |
| 42 | 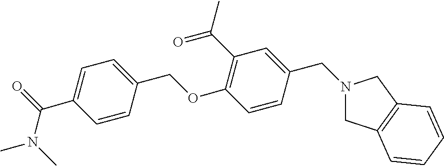 [SMILES: CC(=O)c1cc(CN2Cc3ccccc3C2)ccc1OCc1ccc(C(=O)N(C)C)cc1] Starting materials: 1-(2-Hydroxy-5- (isoindolin-2-ylmethyl)phenyl)- ethenone and 4-(Chloromethyl)-N,N- dimethylbenzamide | ¹H NMR (Chloroform-d) δ: 8.28 (br s, 2H), 7.65-7.77 (m, 2H), 7.46-7.51 (m, 4H), 7.30-7.34 (m, 2H), 7.22-7.26 (m, 2H), 7.09 (br d, 1H), 5.16-5.24 (m, 2H), 4.43 (s, 4H), 4.19-4.31 (m, 2H), 3.13 (br s, 3H), 3.01 (s, 3H), 2.62 (s, 3H). LC-MS: m/z 429.6 (M + H)⁺. |
| 43 | 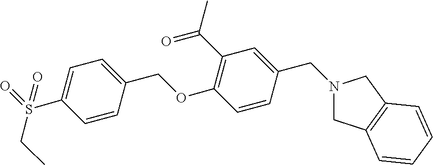 [SMILES: CCS(=O)(=O)c1ccc(COc2ccc(CN3Cc4ccccc4C3)cc2C(C)=O)cc1] Starting materials: 1-(2-Hydroxy-5- (isoindolin-2-ylmethyl)phenyl)- ethenone and 1-(Bromomethyl)-4- (ethylsulfonyl)benzene | ¹H NMR (Chloroform-d) δ: 7.94-7.99 (m, 2H), 7.75 (d, 1H), 7.66 (d, 2H), 7.60 (m, 1H), 7.18-7.24 (m, 4H), 6.99 (d, 1H), 5.27 (s, 2H), 4.03 (s, 4H), 3.96 (s, 2H), 3.11-3.20 (m, 2H), 2.63 (s, 3H), 1.27-1.34 (m, 3H). LC-MS: m/z 450.4 (M + H)⁺. |
| 44 | 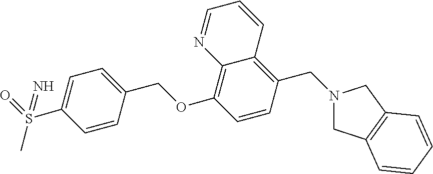 [SMILES: CS(=N)(=O)c1ccc(COc2ccc(CN3Cc4ccccc4C3)c3cccnc23)cc1] Starting materials: 5-(Isoindolin-2-yl- methyl)quinolin-8-ol and 1-(Chloro- methyl)-4-(S-methylsulfonimidoyl)- benzene | ¹H NMR (Chloroform-d) δ: 8.99 (m, 1H), 8.74 (m, 1H), 8.00-8.07 (m, 2H), 7.73 (d, 2H), 7.44-7.50 (m, 1H), 7.38 (d, 1H), 7.13-7.19 (m, 4H), 6.91 (d, 1H), 5.52 (s, 2H), 4.22 (s, 2H), 3.91- 3.94 (m, 4H), 3.12 (s, 3H), 2.01 (s, 1H). LC-MS: m/z 444.4 (M + H)⁺. |
| 45 | 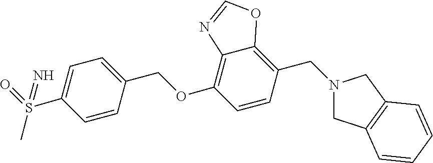 [SMILES: CS(=N)(=O)c1ccc(COc2ccc(CN3Cc4ccccc4C3)c3ocnc23)cc1] Starting materials: 4-(Isoindolin-2-yl- methyl)benzo[d]oxazol-7-ol and 1- (Chloromethyl)-4-(S-methylsulfon- imidoyl)benzene | ¹H NMR (Chloroform-d) δ: 8.11 (s, 1H), 8.03-8.07 (m, 2H), 7.68 (d, 2H), 7.38 (d, 1H), 7.16 (s, 4H), 6.93 (d, 1H), 5.40 (s, 2H), 4.26 (s, 2H), 4.00 (s, 4H), 3.11 (s, 3H), 1.45 (br d, J = 8.74 Hz, 1H). LC-MS: m/z 435.2 (M + H)⁺. |

### Example 3

### 4-(Isoindolin-2-ylmethyl)-7-((1-(methylsulfonyl)piperidin-4-yl)methoxy)-benzo[d]oxazole (Compound 46)

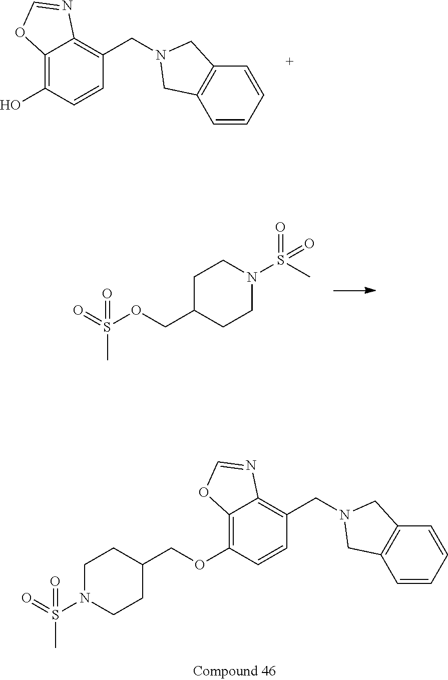 [SMILES: Oc1ccc(CN2Cc3ccccc3C2)c2ncoc12 ; CS(=O)(=O)OCC1CCN(S(C)(=O)=O)CC1 ; CS(=O)(=O)N1CCC(COc2ccc(CN3Cc4ccccc4C3)c3ncoc23)CC1]

To a solution of 4-(isoindolin-2-ylmethyl)benzo[d]oxazol-7-ol (0.10 g, 0.38 mmol) in DMSO (4 ml) was added 4-(methanesulphonyloxymethyl)-1-methane-sulphonylpiperidine (0.11 g, 0.41 mmol), tris(2-(2-methoxyethoxy)ethyl)amine (5.23 μl, 0.02 mmol) and Cs₂CO₃ (0.21 g, 0.64 mmol) under nitrogen. The reaction mixture was stirred at 80° C. for 45 min. Water (10 ml) was added, and the product was extracted with EtOAc. The combined extracts were washed with water, dried with Na₂SO₄, filtered and evaporated. The crude product was purified by chromatography to afford 124.0 mg of the title compound. ¹H NMR (400 MHz, Chloroform-d) δ: 1.98-2.09 (m, 4H), 2.68-2.80 (m, 4H), 2.81 (s, 3H), 3.87-3.93 (m, 2H), 4.00 (s, 4H), 4.26 (s, 2H), 6.88 (d, J=8.25 Hz, 1H), 7.16 (s. 4H), 7.26 (s, 1H), 7.37 (d, J=8.25 Hz, 1H), 8.08 (s, 1H). LC-MS: m/z 442.5 (M+H)⁺.

The following compounds were prepared according to the procedures described for Compound 46. The compound number, characterization data and starting materials are indicated on the table.

| No | Structure and starting material | 1H NMR/LC-MS |
|---|---|---|
| 47 | 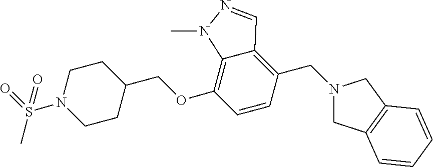 Starting materials: 4-(Isoindolin-2-yl- methyl)-1-methyl-1H-indazol-7-ol trifluoroacetate and 4-(Methane- sulphonyloxymethyl)-1-methane- sulphonylpiperidine | ¹H NMR (400 MHz, Chloroform-d) δ:1.45 (br d, J = 8.74 Hz, 1 H), 1.52- 1.66 (m, 4 H), 1.82-1.94 (m, 4 H), 2.72-2.83 (m, 7 H), 3.81 (br d, J = 11.43 Hz, 2 H), 3.89-4.04 (m, 6 H), 4.12 (s, 2 H), 4.31 (s, 3 H), 6.62 (d, J = 7.64 Hz, 1 H), 7.00 (d, J = 7.64 Hz, 1 H), 7.13-7.19 (m, 4 H), 8.11 (s, 1 H). LC-MS: m/z 442.5 (M + H)⁺. |
| 48 | 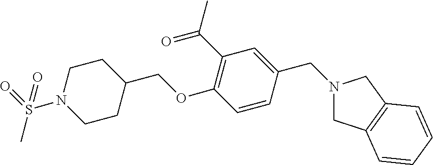 [SMILES: CC(=O)c1cc(CN2Cc3ccccc3C2)ccc1OCC1CCN(S(C)(=O)=O)CC1] Starting materials: 1-(2-Hydroxy-5- (isoindolin-2-ylmethyl)phenyl)- ethenone and 4-(Methanesulphonyl- oxymethyl)-1-methanesulphonyl- piperidine | ¹H NMR (Chloroform-d) δ: 7.71 (d, 1H), 7.54 (m, 1H), 7.15-7.18 (m, 4H), 6.92 (d, 1H), 3.94-3.97 (m, 2H), 3.91 (s, 3H), 3.92-3.87 (m, 2H), 3.86 (s, 2H), 2.80 (s, 3H), 2.73 (m, 2H), 2.62 (s, 3H), 1.96-2.03 (m, 4H), 1.48-1.60 (m, 2H). LC-MS: m/z 444.2 (M + H)⁺. |
| 49 | 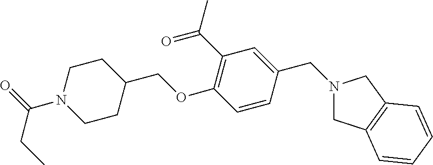 [SMILES: CCC(=O)N1CCC(COc2ccc(CN3Cc4ccccc4C3)cc2C(C)=O)CC1] Starting materials: 1-(2-Hydroxy-5- (isoindolin-2-ylmethyl)phenyl)- ethenone and (1-Propionylpiperidin-4- yl)methyl 4-methylbenzenesulfonate | ¹H NMR (Chloroform-d) δ: 7.67-7.75 (m, 2H), 7.20-7.33 (m, 4H), 7.00 (d, 1H), 4.73 (br d, 2H), 4.33 (s, 4H), 4.18 (s, 2H), 3.87-4.04 (m, 4H), 2.63 (s, 3H), 2.32-2.42 (m, 2H), 2.07-2.26 (m, 1H), 1.82-2.01 (m, 2H), 1.24-1.43 (m, 2H), 1.16 (m, 3H). LC-MS: m/z 421.4 (M + H)⁺. |
| 50 | 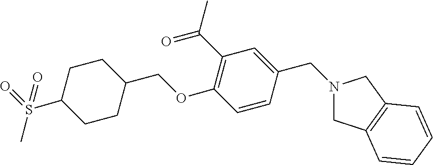 [SMILES: CC(=O)c1cc(CN2Cc3ccccc3C2)ccc1OCC1CCC(S(C)(=O)=O)CC1] Starting materials: 1-(2-Hydroxy-5- (isoindolin-2-ylmethyl)phenyl)- ethenone and (4-(Methylsulfonyl)- cyclohexyl)methyl 4-methylbenzene- sulfonate | ¹H NMR (Chloroform-d) δ: 7.72 (br s, 2H), 7.16-7.26 (m, 4H), 7.01 (br d, 1H), 4.24 (br s, 4H), 4.10 (br s, 2H), 4.06 (d, 2H), 2.96-3.05 (m, 1H), 2.88 (s, 3H), 2.63 (s, 3H), 2.25 (br s, 1H), 2.15 (br d, 1H), 2.02-1.35 (m, 8H). LC-MS: m/z 442.5 (M + H)⁺. |
| 51 | 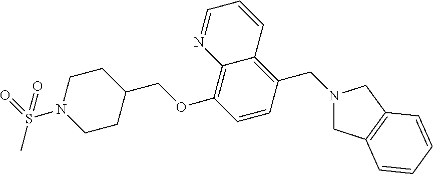 [SMILES: CS(=O)(=O)N1CCC(COc2ccc(CN3Cc4ccccc4C3)c3cccnc23)CC1] Starting materials: 5-(Isoindolin-2-yl- methyl)quinolin-8-ol and 4-(Methane- sulphonyloxymethyl)-1-methane- sulphonylpiperidine | ¹H NMR (Chloroform-d) δ: 8.89-8.98 (m, 1H), 8.72 (m, 1H), 7.38-7.48 (m, 2H), 7.11-7.20 (m, 4H), 6.91-7.02 (m, 1H), 4.23 (s, 2H), 4.10 (d, 2H), 3.84- 3.97 (m, 6H), 2.81 (s, 3H), 2.71-2.79 (m, 2H), 2.29 (br d, 1H), 2.17 (br d, 2H), 1.55-1.53 (m, 2H). LC-MS: m/z 452.3 (M + H)⁺. |

### Example 4

### 4-((2-Amino-4-(isoindolin-2-ylmethyl)phenoxy)methyl)-N,N-dimethyl-benzamide (Compound 52)

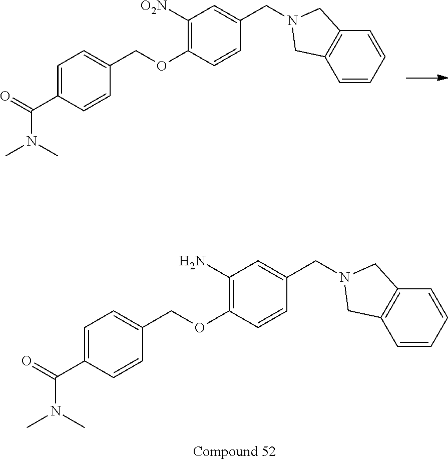 [SMILES: CN(C)C(=O)c1ccc(COc2ccc(CN3Cc4ccccc4C3)cc2[N+](=O)[O-])cc1 ; CN(C)C(=O)c1ccc(COc2ccc(CN3Cc4ccccc4C3)cc2N)cc1]

The solution of 4-((4-(isoindolin-2-ylmethyl)-2-nitrophenoxy)methyl)-N,N-dimethylbenzamide (0.200 g, 0.46 mmol), ammonium chloride (62.0 mg 1.16 mmol) and zinc (0.30 g, 4.64 mmol) was stirred at 65° C. for 1 h. The mixture was filtered, and the precipitate was washed with methanol. The filtrate was concentrated under reduced pressure and the residue was purified by reverse phase chromatography to afford 89.0 mg of the title compound. ¹H NMR (400 MHz, DMSO-d₆) δ: 2.83-3.02 (m, 6H), 3.34-3.43 (m, 2H), 3.65-3.70 (m, 2H), 3.74-3.80 (m, 4H), 5.07-5.13 (m, 2H), 6.49 (dd, J=8.09, 2.07 Hz, 1H), 6.73 (d, J=2.07 Hz, 1H), 6.79-6.85 (m, 1H), 7.13-7.22 (m, 4H), 7.37-7.47 (m, 2H), 7.54 (d, J=8.41 Hz, 2H). LC-MS: m/z 402.4 (M+H)⁺.

### Example 5

### 4-((4-(Isoindolin-2-ylmethyl)-2-(methylsulfonamido)phenoxy)methyl)-N,N-dimethylbenzamide (Compound 53)

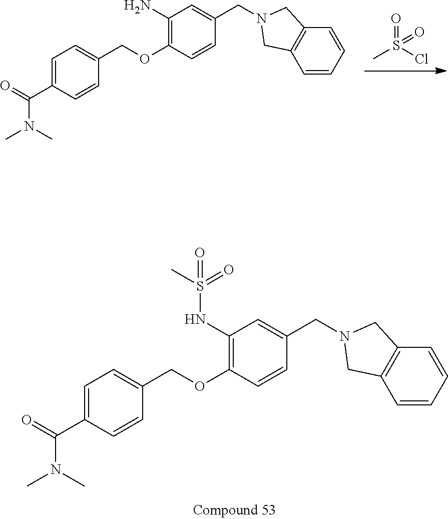

To the solution of 4-((2-amino-4-(isoindolin-2-ylmethyl)phenoxy)methyl)-N,N-dimethylbenzamide (0.23 g, 0.56 mmol) and methane sulfonyl chloride (0.045 ml, 0.56 mmol) in DCM (10 ml) at 0° C. was added pyridine (0.06 ml, 0.68 mmol). The mixture was stirred at RT for 12 h. Water (10 ml) was added followed by 1 M NaOH until pH 8.

The product was extracted by DCM, washed with water, dried with Na₂SO₄ and filtrated. The filtrate was concentrated under reduced pressure and the residue was purified by reverse phase chromatography to afford 26.5 mg of the title compound. ¹H NMR (Chloroform-d) δ: 7.57 (d, 1H), 7.40-7.53 (m, 4H), 7.18 (s, 4H), 6.95 (d, 1H), 6.78 (s, 1H), 5.13 (s, 2H), 3.93 (s, 4H), 3.86 (s, 2H), 3.13 (br s, 3H), 3.03 (s, 3H), 2.97 (s, 3H). LC-MS: m/z 480.8 (M+H)⁺.

### Example 6

### 5-(Isoindolin-2-ylmethyl)-2-((4-(methylsulfonyl)benzyl)oxy)benzamide (Compound 54)

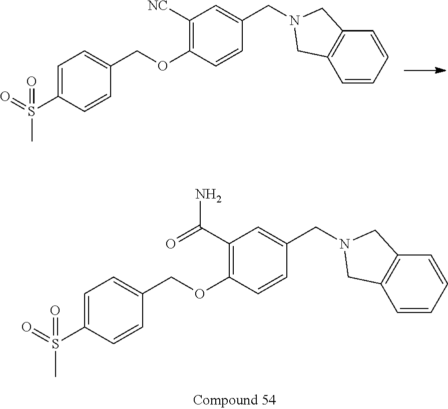 [SMILES: CS(=O)(=O)c1ccc(COc2ccc(CN3Cc4ccccc4C3)cc2C#N)cc1 ; CS(=O)(=O)c1ccc(COc2ccc(CN3Cc4ccccc4C3)cc2C(N)=O)cc1]

To the solution of 5-(isoindolin-2-ylmethyl)-2-((4-(methylsulfonyl)benzyl)oxy)-benzonitrile (80.0 mg, 0.19 mmol) in DMSO (2 ml) was added K₂CO₃ (0.11 g, 0.76 mmol) and hydrogen peroxide (35 wt-%) in water (0.54 ml, 0.65 mmol). The mixture was stirred at 60° C. for 1 h. The reaction mixture was concentrated under reduced pressure. Water (10 ml) was added and the obtained precipitate was filtered, washed with water and dried in oven followed by purification by reverse phase chromatography to afford 7.0 mg of the title compound. ¹H NMR (400 MHz, DMSO-d₆) δ: 3.78-3.85 (m, 9H), 5.39 (s, 2H), 7.12-7.25 (m, 5H), 7.43 (dd, J=8.38, 2.16 Hz, 1H), 7.55 (br s, 1H), 7.66 (br s, 1H), 7.77 (d, J=8.22 Hz, 3H), 7.97 (d, J=8.34 Hz, 2H). LC-MS: m/z 437.5 (M+H)⁺.

### Example 7

### 4-((4-(Isoindolin-2-ylmethyl)-2-(2,2,2-trifluoroacetamido)phenoxy)methyl)-N,N-dimethylbenzamide (Compound 55)

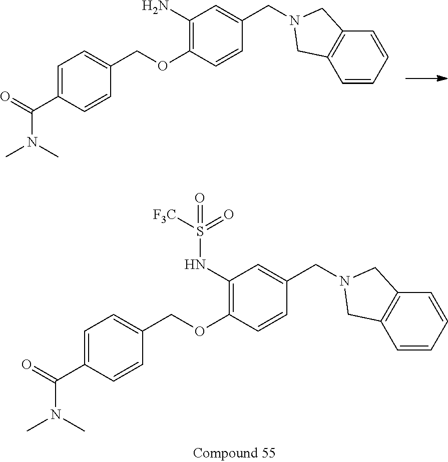 [SMILES: CN(C)C(=O)c1ccc(COc2ccc(CN3Cc4ccccc4C3)cc2N)cc1 ; CN(C)C(=O)c1ccc(COc2ccc(CN3Cc4ccccc4C3)cc2NS(=O)(=O)C(F)(F)F)cc1]

To the solution of 4-((2-amino-4-(isoindolin-2-ylmethyl)phenoxy)methyl)-N,N-dimethylbenzamide (44.0 mg, 0.11 mmol) and trifluoroacetic anhydride (0.02 ml, 0.13 mmol) in DCM (10 ml) at 0° C. was added pyridine (0.13 ml, 1.64 mmol). The mixture was stirred at RT for 1 h. Water (10 ml) was added followed by 1 M NaOH until pH 8. The product was extracted by DCM, washed with water, dried with Na₂SO₄ and filtered. The filtrate was concentrated under reduced pressure and the residue was purified by reverse phase chromatography to afford 21.0 mg of the title compound. ¹H NMR (Chloroform-d) δ: 7.57 (d, 1H), 7.40-7.57 (m, 3H), 7.18 (s, 4H), 6.95 (d, 1H), 5.13 (s, 2H), 3.93 (s, 4H), 3.86 (s, 2H), 3.13 (br s, 3H), 3.03 (s, 3H), 2.97 (s, 3H). LC-MS: m/z 480.8 (M+H)⁺.

### Example 8

### (2-(4-((1-(Methylsulfonyl)piperidin-4-yl)methoxy)-3-(trifluoromethyl)benzyl)-isoindoline (Compound 56)

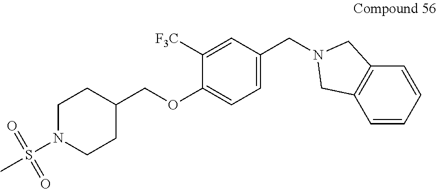 [SMILES: CS(=O)(=O)N1CCC(COc2ccc(CN3Cc4ccccc4C3)cc2C(F)(F)F)CC1]

### a) Methyl 4-((1-(methylsulfonyl)piperidin-4-yl)methoxy)-3-(trifluoromethyl)-benzoate

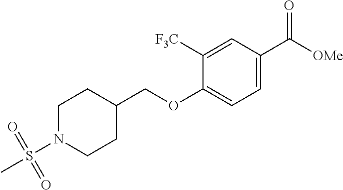 [SMILES: COC(=O)c1ccc(OCC2CCN(S(C)(=O)=O)CC2)c(C(F)(F)F)c1]

To a mixture of methyl 4-hydroxy-3-(trifluoromethyl)benzoate (0.33 g, 1.50 mmol) and (1-(methylsulfonyl)piperidin-4-yl)methyl methanesulfonate (0.448 g, 1.65 mmol) in dry DMSO (4.5 ml) was added potassium carbonate (0.456 g, 3.30 mmol). The mixture was stirred at 100° C. until the reaction was completed (analysed by LC-MS). Water was added to the cooled reaction mixture followed by stirring at RT. The precipitated product was filtered, washed with water and dried under vacuum to afford the title compound (0.54 g). LC-MS: m/z=396.5 (M+H)⁺.

### b) (4-((1-(Methylsulfonyl)piperidin-4-yl)methoxy)-3-(trifluoromethyl)phenyl)-methanol

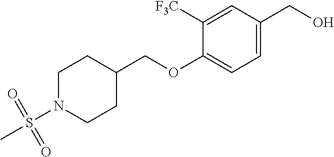 [SMILES: CS(=O)(=O)N1CCC(COc2ccc(CO)cc2C(F)(F)F)CC1]

To a cooled (0-5° C.) mixture of methyl 4-((1-(methylsulfonyl)piperidin-4-yl)-methoxy)-3-(trifluoromethyl)benzoate (0.54 g, 1.366 mmol) in dry THF (6 ml) was added lithium borohydride (2 M in THF, 1.7 ml, 3.40 mmol). The mixture was stirred at RT for 2.5 h and then at 60° C. until the reaction was completed (analysed by LC-MS). The reaction mixture was cooled to 0-5° C., treated with dry acetone (6 ml), stirred at RT and evaporated to dryness. To the residue was added water and saturated NH₄Cl-solution and then extracted with EtOAc (2×). Combined organic phases were washed with water and brine, dried over anhydrous Na₂SO₄, filtered and evaporated to afford the title compound (0.57 g). LC-MS: m/z=368.3 (M+H)⁺.

### c) 4-((4-(Chloromethyl)-2-(trifluoromethyl)phenoxy)methyl)-1-(methylsulfonyl)piperidine

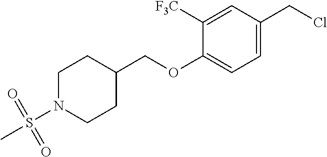 [SMILES: CS(=O)(=O)N1CCC(COc2ccc(CCl)cc2C(F)(F)F)CC1]

To a mixture of (4-((1-(methylsulfonyl)piperidin-4-yl)methoxy)-3-(trifluoro-methyl)phenyl)methanol (0.57 g, 1.365 mmol) in dry DCM (7 ml) containing catalytic amount of DMF was added thionyl chloride (0.20 ml, 2.74 mmol). The mixture was stirred at RT until the reaction was completed (analysed by LC-MS). Solvents were evaporated, dry DCM was added and the evaporation repeated. Drying under vacuum afforded the title compound (0.516 g). LC-MS: m/z=386.4 (M+H)⁺.

### d) 2-(4-((1-(Methylsulfonyl)piperidin-4-yl)methoxy)-3-(trifluoromethyl)benzyl)-isoindoline

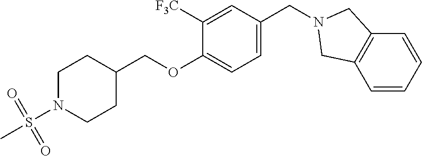 [SMILES: CS(=O)(=O)N1CCC(COc2ccc(CN3Cc4ccccc4C3)cc2C(F)(F)F)CC1]

A mixture of 4-((4-(chloromethyl)-2-(trifluoromethyl)phenoxy)methyl)-1-(methylsulfonyl)piperidine (0.123 g, 0.3320 mmol), isoindoline (0.042 g, 0.352 mmol) and N,N-diisopropylamine (DIPEA) (0.15 ml, 0.861 mmol) in dry DMSO (1.5 ml) was stirred at 60° C. until the reaction was completed (analysed by LC-MS). Water and EtOAc were added to the cooled reaction mixture and and phases were separated.

Aqueous phase was extracted with EtOAc and the combined organic extracts were washed with water and brine, dried and evaporated. Crude product was purified with reverse phase flash chromatography to afford the title compound (0.033 g). ¹H NMR (400 MHz, CDCl₃): δ 8.29 (s, 1H), 7.65 (d, 1H), 7.60 (s, 1H), 7.18-7.31 (m, 3H), 6.99 (d, 1H), 4.19 (s, 4H), 4.09 (s, 2H), 3.83-3.97 (m, 4H), 2.81 (s, 3H), 2.75 (td, 2H), 1.90-2.06 (m, 3H), 1.45-1.60 (m, 2H). LC-MS: m/z=513.3 (M−H)⁺.

### Example 9

### 2-(3-Cyclopropyl-4-((1-(methylsulfonyl)piperidin-4-yl)methoxy)benzyl)iso-indoline (Compound 57)

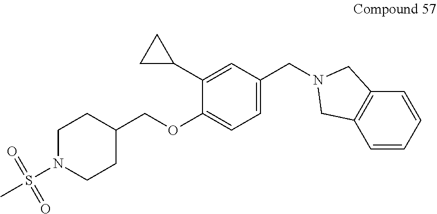 [SMILES: CS(=O)(=O)N1CCC(COc2ccc(CN3Cc4ccccc4C3)cc2C2CC2)CC1]

### a) 3-Bromo-4-((1-(methylsulfonyl)piperidin-4-yl)methoxy)benzaldehyde

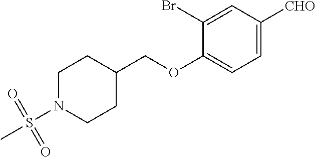 [SMILES: CS(=O)(=O)N1CCC(COc2ccc(C=O)cc2Br)CC1]

The compound was prepared according to the procedure of Example 8 (Step a) starting from 3-bromo-4-hydroxybenzaldehyde (0.201 g, 1.00 mmol), (1-(methyl-sulfonyl)piperidin-4-yl)methyl methanesulfonate (0.298 g, 1.10 mmol) and potassium carbonate (0.304 g, 2.20 mmol) in dry DMSO (3.0 ml). Yield 0.39 g. LC-MS: m/z=376.1/378.1 (M+H)⁺.

### b) 3-Cyclopropyl-4-((1-(methylsulfonyl)piperidin-4-yl)methoxy)benzaldehyde

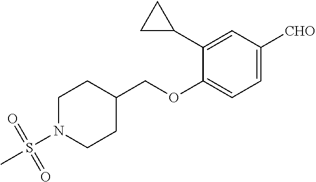 [SMILES: CS(=O)(=O)N1CCC(COc2ccc(C=O)cc2C2CC2)CC1]

To a mixture of 3-bromo-4-((1-(methylsulfonyl)piperidin-4-yl)methoxy)-benzaldehyde (0.376 g, 1.00 mmol), potassium phosphate (0.637 g, 3.00 mmol), cyclopropylboronic acid (0.129 g, 1.50 mmol), toluene (5.0 ml) and water (0.35 ml) under nitrogen atmosphere was added dichloro[1,1′-bis(diphenylphosphino)ferrocene]-palladium dichloromethane adduct (0.041 g, 0.050 mmol). The mixture was stirred at 100° C. until reaction was completed (analysed by LC-MS). Cooled reaction mixture was diluted with EtOAc and filtered through a short plug of Celite. Filtrate was washed with water and brine, dried and evaporated. Crude product was purified with flash chromatography to afford the title compound. Yield 0.25 g. LC-MS: m/z=338.4 (M+H)⁺.

### c) (3-Cyclopropyl-4-((1-(methylsulfonyl)piperidin-4-yl)methoxy)phenyl)-methanol

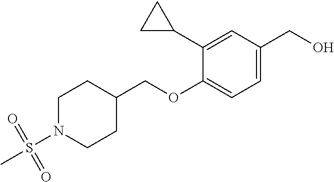 [SMILES: CS(=O)(=O)N1CCC(COc2ccc(CO)cc2C2CC2)CC1]

To a mixture of 3-cyclopropyl-4-((1-(methylsulfonyl)piperidin-4-yl)methoxy)-benzaldehyde (0.25 g, 0.741 mmol) in methanol (3.0 ml) was added sodium borohydride (0.112 g, 2.964 mmol) in small portions. The mixture was stirred at RT overnight. Methanol was evaporated and residue was treated with aqueous NH₄Cl and extracted with EtOAc. Organic phase was washed with water and brine, dried and evaporated. Crude product was purified with flash chromatography to afford the title compound. Yield 0.15 g. LC-MS: m/z=312.5 (M−H₂O+H)⁺.

### d) 4-((4-(Chloromethyl)-2-cyclopropylphenoxy)methyl)-1-(methylsulfonyl)-piperidine

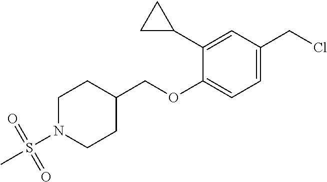 [SMILES: CS(=O)(=O)N1CCC(COc2ccc(CCl)cc2C2CC2)CC1]

The compound was prepared according to the procedure of Example 8(c) starting from (3-cyclopropyl-4-((1-(methylsulfonyl)piperidin-4-yl)methoxy)phenyl)methanol (0.15 g, 0.442 mmol) and thionyl chloride (0.097 ml, 1.326 mmol) in dry DCM (2.5 ml). Yield 0.17 g. ¹H NMR (400 MHz, CDCl₃): δ 7.13 (dd, 1H), 6.87 (d, 1H), 6.76 (d, 1H), 4.52 (s, 2H), 3.84-3.93 (m, 4H), 2.80 (s, 3H), 2.72 (td, 2H), 2.07-2.16 (m, 1H), 1.92-2.04 (m, 3H), 1.50-1.64 (m, 2H), 0.89-0.96 (m, 2H), 0.62-0.69 (m, 2H).

### e) 2-(3-Cyclopropyl-4-((1-(methylsulfonyl)piperidin-4-yl)methoxy)benzyl)-isoindoline

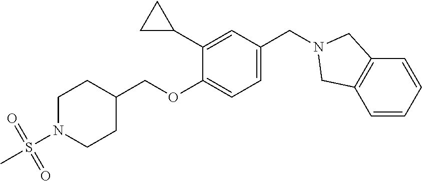 [SMILES: CS(=O)(=O)N1CCC(COc2ccc(CN3Cc4ccccc4C3)cc2C2CC2)CC1]

The compound was prepared according to the procedure of Example 8(d) starting from 4-((4-(chloromethyl)-2-cyclopropylphenoxy)methyl)-1-(methylsulfonyl)piperidine (0.085 g, 0.238 mmol, isoindoline (0.030 ml, 0.261 mmol) and DIPEA (0.10 ml, 0.574 mmol) in dry DMSO (1.5 ml). Yield 0.007 g. 7.14-7.22 (m, 4H), ¹H NMR (400 MHz, CDCl₃): δ 7.08 (dd, 1H), 6.88 (d, 1H), 6.80 (d, 1H), 3.88 (d, 2H), 3.77 (s, 4H), 3.72 (s, 2H), 3.57-3.64 (m, 2H), 2.86 (s, 3H), 2.70-2.78 (m, 2H), 2.08-2.15 (m, 1H), 1.86-1.95 (m, 3H), 1.35-1.46 (m, 2H), 0.85-0.91 (m, 2H), 0.57 (m, 2H). LC-MS: m/z=441.9 (M+H)⁺.

### Example 10

### 1-(5-(Isoindolin-2-ylmethyl)-2-((1-(methylsulfonyl)piperidin-4-yl)methoxy)-phenyl)ethan-1-ol (Compound 58)

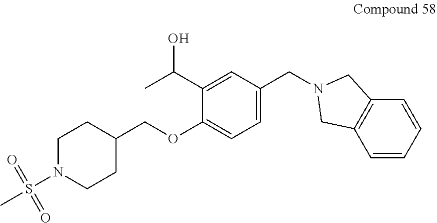 [SMILES: CC(O)c1cc(CN2Cc3ccccc3C2)ccc1OCC1CCN(S(C)(=O)=O)CC1]

To a mixture of 1-(5-(isoindolin-2-ylmethyl)-2-((1-(methylsulfonyl)piperidin-4-yl)methoxy)phenyl)ethan-1-one (Compound 48) (0.034 g, 0.077 mmol) in methanol (2.0 ml) was added sodium borohydride (15.8 mg, 0.418 mmol). The mixture stirred at RT until reaction was completed (analysed by LC-MS). Solvent was evaporated, saturated NaHCO₃ and DCM were added and phases separated. Aqueous phase was extracted with DCM and combined organic extracts were dried and evaporated. Crude product was purified with reverse phase flash chromatography to afford the title compound. Yield 0.023 g. ¹H NMR (400 MHz, CDCl₃): δ 7.47 (s, 1H), 7.19-7.24 (m, 2H), 7.13-7.19 (m, 3H), 6.87 (d, 1H), 5.00 (s, 2H), 3.87-3.92 (m, 1H), 3.82-3.87 (m, 1H), 3.80 (s, 4H), 3.79 (s, 2H), 3.56-3.64 (m, 2H), 2.86 (s, 3H), 2.75 (t, 2H), 1.82-1.93 (m, 3H), 1.34-1.45 (m, 2H), 1.28 (d, 3H). LC-MS: m/z=445.8 (M+H)⁺.

### Abbreviations

DCM—Dichloromethane
DIPEA—N,N-diisopropylamine
DMF—N,N-Dimethylformamide
DMSO—Dimethylsulfoxide
LC-MS—Liquid chromatography-mass spectrometry
RT—Room temperature
THF—Tetrahydrofuran

### EXPERIMENTS

### Experiment 1. CYP11A1 Inhibition

The ability of the test compounds to inhibit conversion of cholesterol to pregnenolone and isocaproic acid was measured by modification of isocaproic acid release assay (IARA) described by Ruangwises et al. (Biology of Reproduction 1991; 45(1):143-50) except that human H295R adrenocortical carcinoma cell line was used as source of enzyme and extraction was done with dextran-coated charcoal suspension (Isomaa, V. et al., Endocrinology 1982; 111(3):833-843). The H295R cell line has been shown to express all the key steroidogenic enzymes. To determine the half maximal inhibitory concentration (IC₅₀) of the test compounds on CYP11A1 inhibition, the cells were treated for three days with increasing concentrations of the test compounds in the presence of 3 nM [24,25-3H]-labelled cholesterol (American Radiolabelled Chemicals).

The final DMSO concentration was 1%. Cell culture medium was extracted with dextran-coated charcoal suspension and the radiolabelled isocaproic acid was determined by mixing 100 μl of supernatant fraction in 200 μl of scintillation fluid (OptiPhase SuperMix, Perkin Elmer). Radioactivity was measured using a Microbeta scintillation counter (1450 MicroBeta Trilux, Wallac). All the test compounds were studied at 10 concentrations in duplicates.

The compounds of the invention were screened in the above mentioned assay and the IC₅₀ values of the compounds are set forth in Table 1 below wherein “A” refers to an IC₅₀ value of less than 150 nM, “B” refers to IC₅₀ value in range of 151 to 350 nM and “C” refers to IC₅₀ value in range of 351 nM to 1500 nM.

TABLE 1

| Group | Compound No. |
|---|---|
| A | 3, 6, 12, 13, 14, 19, 20, 21, 22, 26, 33, 34, 35, 38, 40, |
| | 41, 42, 43, 44, 47, 48, 51 and 54 |
| B | 4, 5, 7, 8, 10, 11, 15, 16, 18, 25, 36, 37, 49, 50 and 56 |
| C | 1, 2, 9, 17, 23, 24, 27, 28, 29, 30, 31, 32, 39, 45, 46, 52, |
| | 53 and 55 |

1. A compound of formula (I) or a tautomer or a pharmaceutically acceptable salt thereof 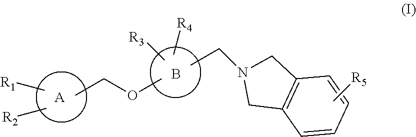
wherein
A is a 3-10 membered carbocyclyl or a 4-12 membered heterocyclyl containing 1-4 heteroatoms selected from the group consisting of O, N and S;
B is phenyl or a 8-12 membered heterocyclyl containing 1-4 heteroatoms selected from the group consisting of O, N and S;
R₁ is hydrogen, halogen, C₁₋₇ alkyl, C₂₋₇ alkenyl, C₃₋₇ cycloalkyl, C₁₋₇ alkoxy, hydroxy, hydroxy C₁₋₇ alkyl, halogen C₁₋₇ alkyl, —X₁—C(O)R₆, —SO₂—R₇, or —S(O)(═NH)(C₁₋₇ alkyl);
with the proviso that when B is phenyl, then R₁ is not hydrogen;
R₂ is hydrogen, C₁₋₇ alkyl, C₂₋₇ alkenyl, C₃₋₇ cycloalkyl, C₁₋₇ alkoxy, halogen or halogen C₁₋₇ alkyl;
R₃ is hydrogen, C₁₋₇ alkyl, C₂₋₇ alkenyl, C₃₋₇ cycloalkyl, hydroxy C₁₋₇ alkyl, halogen C₁₋₇ alkyl, C₁₋₇ alkoxy C₁₋₇ alkyl, nitro, cyano, amino, halogen, —X₂—C(O)R₈, —X₃—NR₉R₁₀ or —OR₁₁;
R₄ is hydrogen, halogen or nitro;
R₅ is hydrogen, halogen, halogen C₁₋₇ alkyl, —S—C₁₋₇ alkyl, —S(O)₂—C₁₋₇ alkyl or oxo;
X₁, X₂ and X₃ are, independently, a bond, C₁₋₇ alkyl or C₂₋₇ alkenyl;
R₆ is C₁₋₇ alkyl, C₁₋₇ alkoxy, or —NR₁₂R₁₃;
R₇ is C₁₋₇ alkyl, C₁₋₇ alkoxy C₁₋₇ alkyl, —NHR₁₄ or a 4-6 membered heterocyclyl optionally substituted with 1-3 C₁₋₇ alkyl groups;
R₈ is C₁₋₇ alkoxy or —NR₁₅R₁₆;
R₉ is hydrogen, C₁₋₇ alkyl, —C(O)—C₁₋₇ alkyl, —C(O)-halogen C₁₋₇ alkyl or —S(O)₂—C₁₋₇ alkyl;
R₁₀ is hydrogen or C₁₋₇ alkyl:
R₁ is hydrogen, C₁₋₇ alkyl, halogen C₁₋₇ alkyl or —C₁₋₇ alkyl-C(O)—NH₂;
R₁₂ is hydrogen, C₁₋₇ alkyl, C₁₋₇ alkoxy C₁₋₇ alkyl, or —C₁₋₇ alkyl-O—C(O)—C₁₋₇ alkyl;
R₁₃ is hydrogen or C₁₋₇ alkyl;
R₁₄ is C₁₋₇ alkyl or C₁₋₇ alkoxy C₁₋₇ alkyl; and
R₁₅ and R₁₆ are, independently, hydrogen or C₁₋₇ alkyl.

2. The compound according to claim 1, wherein B is phenyl or any one of the following groups: 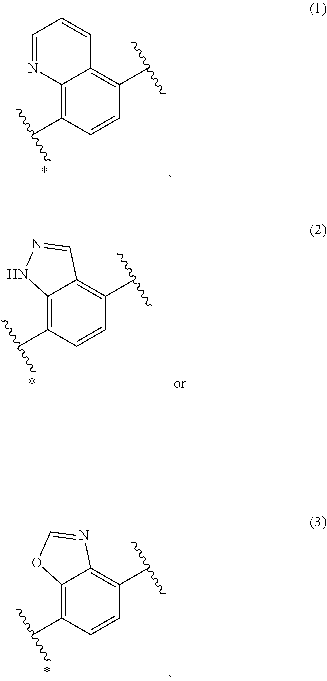 [SMILES: *c1ccc(*)c2ncccc12 ; *c1ccc(*)c2[nH]ncc12 ; *c1ccc(*)c2ocnc12]
wherein the asterisk denotes the point of attachment to oxygen atom.

3. (canceled)

4. The compound according to claim 1, wherein A is a 5-6 membered carbocyclic ring or a 5-6 membered heterocyclic ring containing 1-2 heteroatoms selected from O, N and S.

5. (canceled)

6. The compound according to claim 5, wherein A is phenyl or piperidinyl.

7. The compound according to claim 1, wherein R₁ is halogen, C₁₋₇ alkyl, C₂₋₇ alkenyl, C₃₋₇ cycloalkyl, C₁₋₇ alkoxy, hydroxy, hydroxy C₁₋₇ alkyl, halogen C₁₋₇ alkyl, —X₁—C(O)R₆, —SO₂—R₇ or —S(O)(═NH)(C₁₋₇ alkyl).

8. (canceled)

9. The compound according to claim 7, wherein R₁ is —X₁—C(O)R₆.

10. The compound according to claim 9, wherein X₁ is a bond.

11. The compound according to claim 9, wherein R₆ is C₁₋₇ alkyl, C₁₋₇ alkoxy or —NR₁₂R₁₃ and R₁₂ is C₁₋₇ alkyl, C₁₋₇ alkoxy C₁₋₇ alkyl or —C₁₋₇ alkyl-O—C(O)—C₁₋₇ alkyl.

12. The compound according to claim 1, wherein R₁ is —SO₂—R₇.

13. The compound according to claim 12, wherein R₇ is C₁₋₇ alkyl, C₁₋₇ alkoxy C₁₋₇ alkyl or —NHR₁₄.

14. The compound according to claim 1, wherein R₂ is hydrogen.

15. (canceled)

16. The compound according to claim 1, wherein R₃ is hydrogen, C₃₋₇ cycloalkyl, hydroxy C₁₋₇ alkyl, halogen C₁₋₇ alkyl, C₁₋₇ alkoxy C₁₋₇ alkyl, nitro, cyano, —X₂—C(O)R₈ or —OR₁₁.

17. The compound according to claim 1, wherein X₂ is a bond.

18. The compound according to claim 1, wherein R₈ is
C₁₋₇ alkoxy.

19. The compound according to claim 1, wherein R₁₁ is C₁₋₇ alkyl or —C₁₋₇ alkyl-C(O)—NH₂.

20. The compound according to claim 1, wherein R₄ is hydrogen or halogen.

21. The compound according to claim 1, wherein R₅ is hydrogen, halogen C₁₋₇ alkyl or —S—C₁₋₇ alkyl.

22. The compound or a tautomer or a pharmaceutically acceptable salt thereof according to claim 1, wherein the compound is
Methyl 4-((4-(isoindolin-2-ylmethyl)-2-methoxyphenoxy) methyl) benzoate (Compound 1);
Methyl 4-((4-(isoindolin-2-ylmethyl)phenoxy)methyl)benzoate (Compound 2);
4-((4-(Isoindolin-2-ylmethyl)-2-methoxyphenoxy)methyl)-N,N-dimethylbenzamide (Compound 3);
4-((2-Chloro-4-(isoindolin-2-ylmethyl)phenoxy)methyl)-N,N-dimethylbenzamide (Compound 4);
4-((2-Hydroxy-4-(isoindolin-2-ylmethyl)phenoxy)methyl)-N,N-dimethylbenzamide (Compound 5);
4-((2-Cyano-4-(isoindolin-2-ylmethyl)phenoxy)methyl)-N,N-dimethylbenzamide (Compound 6);
Methyl 2-((4-(dimethylcarbamoyl)benzyl)oxy)-5-(isoindolin-2-ylmethyl)benzoate (Compound 7);
4-((2-Ethoxy-4-(isoindolin-2-ylmethyl)phenoxy)methyl)-N,N-dimethylbenzamide (Compound 8);
Methyl (E)-3-(4-((4-(isoindolin-2-ylmethyl)-2-methoxyphenoxy)methyl)phenyl)acrylate (Compound 9);
4-((4-(Isoindolin-2-ylmethyl)-2-(methoxymethyl)phenoxy)methyl)-N,N-di-methylbenzamide (Compound 10);
4-((4-(Isoindolin-2-ylmethyl)-2-(2,2,2-trifluoroethoxy)phenoxy)methyl)-N,N-dimethylbenzamide (Compound 11);
2-(3-Methoxy-4-((4-(methylsulfonyl)benzyl)oxy)benzyl)isoindoline (Compound 12);
4-((4-(Isoindolin-2-ylmethyl)-2-methoxyphenoxy)methyl)-N-(2-methoxyethyl)benzene-sulfonamide (Compound 13);
4-((2-(2-Amino-2-oxoethoxy)-4-(isoindolin-2-ylmethyl)phenoxy)methyl)-N,N-dimethylbenzamide (Compound 14);
4-((2-Allyl-4-(isoindolin-2-ylmethyl)phenoxy)methyl)-N,N-dimethylbenzamide (Compound 15);
5-(Isoindolin-2-ylmethyl)-2-((4-(methylsulfonyl)benzyl)oxy)benzonitrile (Compound 16);
5-(Isoindolin-2-ylmethyl)-N,N-dimethyl-2-((4-(methylsulfonyl)benzyl)oxy)benzamide (Compound 17);
2-(3-(Methoxymethyl)-4-((4-(methylsulfonyl)benzyl)oxy)benzyl)isoindoline (Compound 18);
5-(Isoindolin-2-ylmethyl)-2-((1-(methylsulfonyl)piperidin-4-yl)methoxy)benzonitrile (Compound 19);
2-(4-((4-((2-Methoxyethyl)sulfonyl)benzyl)oxy)-3-(methoxymethyl)benzyl)isoindoline (Compound 20);
2-(2-Fluoro-3-methoxy-4-((4-(methylsulfonyl)benzyl)oxy)benzyl)isoindoline (Compound 21);
2-(3-(Methoxymethyl)-4-((1-(methylsulfonyl)piperidin-4-yl)methoxy)benzyl)isoindoline (Compound 22);
4-((4-(Isoindolin-2-ylmethyl)-2-methoxy-3-nitrophenoxy)methyl)-N-isopropylpiperidine-1-carboxamide (Compound 23);
4-((3-Fluoro-4-(isoindolin-2-ylmethyl)phenoxy)methyl)-N,N-dimethylbenzamide (Compound 24);
4-((3,5-Difluoro-4-(isoindolin-2-ylmethyl)phenoxy)methyl)-N,N-dimethylbenzamide (Compound 25);
4-((2,5-Difluoro-4-(isoindolin-2-ylmethyl)phenoxy)methyl)-N,N-dimethylbenzamide (Compound 26);
4-((2,3-Difluoro-4-(isoindolin-2-ylmethyl)phenoxy)methyl)-N,N-dimethylbenzamide (Compound 27);
4-((2-Fluoro-4-(isoindolin-2-ylmethyl)-6-methoxyphenoxy)methyl)-N,N-di-methylbenzamide (Compound 28);
4-((4-(Isoindolin-2-ylmethyl)-2-(trifluoromethoxy)phenoxy)methyl)-N,N-dimethylbenzamide (Compound 29);
N,N-Dimethyl-4-((4-((1-oxoisoindolin-2-yl)methyl)-2-(trifluoromethoxy)phenoxy)-methyl)benzamide (Compound 30);
4-((4-(Isoindolin-2-ylmethyl)-3-methoxyphenoxy)methyl)-N,N-dimethylbenzamide (Compound 31);
4-((2-Methoxy-4-((5-(methylsulfonyl)isoindolin-2-yl)methyl)phenoxy)methyl)-N,N-dimethylbenzamide (Compound 32);
5-(isoindolin-2-ylmethyl)-2-((4-(S-methylsulfonimidoyl)benzyl)oxy)benzonitrile (Compound 33);
4-((2-Methoxy-4-((5-(methylthio)isoindolin-2-yl)methyl)phenoxy)methyl)-N,N-dimethylbenzamide (Compound 34);
4-((4-(Isoindolin-2-ylmethyl)-2-methoxyphenoxy)methyl)-N-(2-methoxyethyl)-N-methylbenzamide (Compound 35);
2-(4-((4-(Isoindolin-2-ylmethyl)-2-methoxyphenoxy)methyl)-N-methylbenzamido)ethyl acetate (Compound 36);
2-(3-(2-Methoxyethyl)-4-((1-(methylsulfonyl)piperidin-4-yl)methoxy)benzyl)isoindoline (Compound 37);
4-(Isoindolin-2-ylmethyl)-7-((1-(methylsulfonyl)piperidin-4-yl)methoxy)-1H-indazole (Compound 38);
Methyl 4-((4-(isoindolin-2-ylmethyl)-2-nitrophenoxy)methyl)benzoate (Compound 39);
4-((4-(isoindolin-2-ylmethyl)-2-nitrophenoxy)methyl)-N,N-dimethylbenzamide (Compound 40);
4-(((5-(Isoindolin-2-ylmethyl)quinolin-8-yl)oxy)methyl)-N,N-dimethylbenzamide (Compound 41);
4-((2-Acetyl-4-(isoindolin-2-ylmethyl)phenoxy)methyl)-N,N-dimethylbenzamide (Compound 42);
1-(2-((4-(Ethylsulfonyl)benzyl)oxy)-5-(isoindolin-2-ylmethyl)phenyl)ethan-1-one (Compound 43);
Imino(4-(((5-(isoindolin-2-ylmethyl)quinolin-8-yl)oxy)methyl)phenyl)(methyl)-λ⁶-sulfanone (Compound 44);
Imino(4-(((7-(isoindolin-2-ylmethyl)benzo[d]oxazol-4-yl)oxy)methyl)phenyl)(methyl)-λ⁶-sulfanone (Compound 45);
4-(Isoindolin-2-ylmethyl)-7-((1-(methylsulfonyl)piperidin-4-yl)methoxy)benzo[d]oxazole (Compound 46);
4-(Isoindolin-2-ylmethyl)-1-methyl-7-((1-(methylsulfonyl)piperidin-4-yl)methoxy)-1H-indazole (Compound 47);
1-(5-(Isoindolin-2-ylmethyl)-2-((1-(methylsulfonyl)piperidin-4-yl)methoxy)phenyl)-ethan-1-one (Compound 48);
1-(4-((2-Acetyl-4-(isoindolin-2-ylmethyl)phenoxy)methyl)piperidin-1-yl)propan-1-one (Compound 49);
1-(5-(Isoindolin-2-ylmethyl)-2-((4-(methylsulfonyl)cyclohexyl)methoxy)phenyl)ethan-1-one (Compound 50);
5-(Isoindolin-2-ylmethyl)-8-((1-(methylsulfonyl)piperidin-4-yl)methoxy)quinoline (Compound 51);
4-((2-Amino-4-(isoindolin-2-ylmethyl)phenoxy)methyl)-N,N-dimethylbenzamide (Compound 52);
4-((4-(Isoindolin-2-ylmethyl)-2-(methylsulfonamido)phenoxy)methyl)-N,N-dimethylbenzamide (Compound 53);
5-(Isoindolin-2-ylmethyl)-2-((4-(methylsulfonyl)benzyl)oxy)benzamide (Compound 54); or
4-((4-(Isoindolin-2-ylmethyl)-2-(2,2,2-trifluoroacetamido)phenoxy)methyl)-N,N-dimethylbenzamide (Compound 55.

23-28. (canceled)

29. A method for the treatment of a steroid receptor dependent condition or disease comprising administering to a subject in need thereof a therapeutically effective amount of a compound of formula (I) or a pharmaceutically acceptable salt thereof 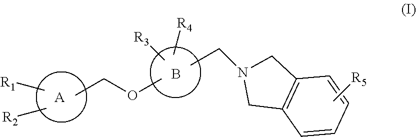
wherein
A is a 3-10 membered carbocyclyl or a 4-12 membered heterocyclyl containing 1-4 heteroatoms selected from the group consisting of O, N and S;
B is phenyl or a 8-12 membered heterocyclyl containing 1-4 heteroatoms selected from the group consisting of O, N and S;
R₁ is hydrogen, halogen, C₁₋₇ alkyl, C₂₋₇ alkenyl, C₃₋₇ cycloalkyl, C₁₋₇ alkoxy, hydroxy, hydroxy C₁₋₇ alkyl, halogen C₁₋₇ alkyl, —X₁—C(O)R₆, —SO₂—R₇, or —S(O)(═NH)(C₁₋₇ alkyl);
with the proviso that when B is phenyl, then R₁ is not hydrogen;
R₂ is hydrogen, C₁₋₇ alkyl, C₂₋₇ alkenyl, C₃₋₇ cycloalkyl, C₁₋₇ alkoxy, halogen or halogen C₁₋₇ alkyl;
R₃ is hydrogen, C₁₋₇ alkyl, C₂₋₇ alkenyl, C₃₋₇ cycloalkyl, hydroxy C₁₋₇ alkyl, halogen C₁₋₇ alkyl, C₁₋₇ alkoxy C₁₋₇ alkyl, nitro, cyano, amino, halogen, —X₂—C(O)R₈, —X₃—NR₉R₁₀ or —OR₁₁;
R₄ is hydrogen, halogen or nitro;
R₅ is hydrogen, halogen, halogen C₁₋₇ alkyl, —S—C₁₋₇ alkyl, —S(O)₂—C₁₋₇ alkyl or oxo;
X₁, X₂ and X₃ are, independently, a bond, C₁₋₇ alkyl or C₂₋₇ alkenyl;
R₆ is C₁₋₇ alkyl, C₁₋₇ alkoxy, or —NR₁₂R₁₃;
R₇ is C₁₋₇ alkyl, C₁₋₇ alkoxy C₁₋₇ alkyl, —NHR₁₄ or a 4-6 membered heterocyclyl optionally substituted with 1-3 C₁₋₇ alkyl groups;
R₈ is C₁₋₇ alkoxy or —NR₁₅R₁₆;
R₉ is hydrogen, C₁₋₇ alkyl, —C(O)—C₁₋₇ alkyl, —C(O)-halogen C₁₋₇ alkyl or —S(O)₂—C₁₋₇ alkyl;
R₁₀ is hydrogen or C₁₋₇ alkyl:
R₁ is hydrogen, C₁₋₇ alkyl, halogen C₁₋₇ alkyl or —C₁₋₇ alkyl-C(O)—NH₂;
R₁₂ is hydrogen, C₁₋₇ alkyl, C₁₋₇ alkoxy C₁₋₇ alkyl, or —C₁₋₇ alkyl-O—C(O)—C₁₋₇ alkyl;
R₁₃ is hydrogen or C₁₋₇ alkyl;
R₁₄ is C₁₋₇ alkyl or C₁₋₇ alkoxy C₁₋₇ alkyl; and
R₁₅ and R₁₆ are, independently, hydrogen or C₁₋₇ alkyl.

30. The method according to claim 29, wherein the steroid receptor dependent condition or disease is cancer.

31. The method according to claim 30, wherein the steroid receptor dependent condition or disease is prostate cancer or breast cancer.

32. The method according to claim 29, wherein a therapeutically effective amount of the compound of formula (I) is administered in addition to a glucocorticoid and/or a mineralocorticoid and, optionally, one or more anti-cancer agent.

33. The method according to claim 29, wherein a therapeutically effective amount of the compound of formula (I) is administered in addition to one or more anti-cancer agents selected from the group consisting of
a non-steroidal androgen receptor antagonist;
a steroidogenesis inhibitor;
a chemotherapeutic agent;
an antiestrogen;
an epigenetic modulator;
an mTOR inhibitor;
an AKT inhibitor;
a radiopharmaceutical;
a GnRH/LHRH analogue;
a PI3K inhibitor; and
a CDK4/6 inhibitor,
for simultaneous, separate or sequential administration.

34. A pharmaceutical composition comprising a compound according to claim 1, together with a pharmaceutically acceptable carrier.

35. A pharmaceutical combination comprising the compound according to claim 1 and at least one additional active ingredient selected from the list consisting of
a glucocorticoid;
a mineralocorticoids;
a non-steroidal androgen receptor antagonist;
a steroidogenesis inhibitor;
a chemotherapeutic agents-agent;
an antiestrogen;
an epigenetic modulator;
an mTOR inhibitor;
an AKT inhibitor;
a radiopharmaceutical;
a GnRH/LHRH analogue;
a PI3K inhibitor; and
a CDK4/6 inhibitor;
for simultaneous, separate or sequential administration.
